(19) United States
(12) Patent Application Publication
(10) Pub. No.: US 2024/0400625 A1
(43) Pub. Date: Dec. 5, 2024

(54) MAMMALIAN CARDIAC REGENERATION

(71) Applicant: Koninklijke Nederlandse Akademie van Wetenschappen, Amsterdam (NL)

(72) Inventors: Jeroen Petrus Wilhelmus Maria Bakkers, Bilthoven (NL); Dennis Eduard Maria De Bakker, Jena (DE)

(21) Appl. No.: 18/697,340

(22) PCT Filed: Oct. 3, 2022
(86) PCT No.: PCT/NL2022/050553

§ 371 Date: Mar. 29, 2024

(30) Foreign Application Priority Data
Oct. 1, 2021 (EP) 21200545.8

(51) Int. Cl. C07K 14/47; A61K 38/00; A61P 9/10; C12N 5/077; C12N 15/86

(52) U.S. Cl. CPC C07K 14/47; A61P 9/10; C12N 5/0657; C12N 15/86; A61K 38/00; C12N 2501/998; C12N 2750/14143

(57) ABSTRACT

The invention relates to methods of treating an individual suffering from a structural cardiac muscle defect, comprising providing cardiomyocytes of at least part of the cardiac muscle of the individual with a high mobility group A (HMGA) protein to thereby promote proliferation of said cardiomyocytes. The invention further relates to an expression construct for functional expression of said HMGA protein, a pharmaceutical composition, comprising HMGA or said expression construct, and to a method of culturing cardiomyocytes in vitro, comprising providing cardiomyocytes with a HMGA protein or the expression construct.

### FIELD

The invention relates to the field of medical treatment. It specifically relates to methods of treatment of an individual suffering from a structural cardiac muscle defect. The invention further relates to pharmaceutical preparations that are suitable for use in these methods.

### 1 INTRODUCTION

Cardiovascular disease remains the biggest cause of death in the western world, including the consequence of myocardial infarction (WHO. 2019. Lancet 7: E1332-E1345). Patients suffering from a myocardial infarction often survive the initial injury but permanently lose millions of heart muscle cells. Indeed, although the heart is able to efficiently repair the injury by formation of a permanent scar almost no new cardiomyocytes are being regenerated (Kretzschmar et al., 2018. PNAS 115: E12245-E12254), causing ischemic heart injury to be a chronic affliction. Since hearts with ischemic injuries will ultimately develop heart failure, the field is in desperate need of treatments focusing on regenerating the lost myocardium and or restoration of the full functionality.

Interestingly, some species have a robust endogenous capacity to regenerate the heart, including the zebrafish (Poss et al., 2002. Science 298:2188-2190). In zebrafish the surviving cardiomyocytes (CMs) adjacent to the injury area, also known as the border zone (BZ), de-differentiate towards an embryo-like state and proliferate to replace the lost cardiomyocytes (Honkoop et al., 2019. ELife 8:1-27; Jopling et al., 2010. Nature 464: 606-609; Kikuchi et al., 2010. Nature 464:601-605; Wu et al., 2015. Develop Cell 36:36-49). In addition. BZ CMs undergo robust changes in their chromatin organization which underlies their regenerative response (Beisaw et al., 2020. Circulation Res 126:1760-1778).

This raises the question why zebrafish BZ CMs are re-entering the cell cycle while mammalian BZ CMs do not. A potential answer could be formed when looking at the intrinsic properties of cardiomyocyte nuclei. While mammalian CMs are mainly polyploid (human) or multinuclear (mice), zebrafish CMs are mononuclear and diploid, which has been shown to be detrimental for efficient proliferation and zebrafish heart regeneration (González-Rosa et al., 2018. Developmental Cell 44: 433-446; Patterson et al., 2017. Nature Gen 49: 1346-1353: Windmueller et al., 2020. Cell Reports 30:3105-3116).

Regardless of these restrictions, a low level of endogenous CM turnover was observed in the adult human heart (Bergmann et al., 2009. Science 324:98-102). In addition, the neonatal mouse heart has been shown to contain the capacity to induce CM proliferation and heart regeneration in a small time-widow after birth (Porrello et al., 2011. Science 331:1078-1080). Together, these observations suggest the mammalian heart could retain a latent capacity to regenerate.

Indeed, studies in mice have shown that inhibition of the HIPPO signaling pathway can unlock the regenerative capacity of the mammalian heart (Heallen et al., 2013. Development 140:4683-4690: Leach et al., 2017. Nature 550:260-264). In addition, overexpression of the constitutively active Erbb2 receptor induces efficient cardiomyocyte proliferation in mice, allowing for the near complete regeneration of injured hearts (D'Uva et al., 2015. Nat Cell Biol 17:627-638).

These studies, among others, report that BZ cardiomyocytes are most prone to proliferate upon stimulation (D'Uva et al., 2015. Nat Cell Biol 17:627-638: Leach et al., 2017. Nature 550:260-264.: Lin et al., 2014. Circulation Res 115:354-363.: Pasumarthi et al., 2005. Circulation Res 96:110-118: Xiang et al., 2016. Circulation 133:1081-1092). Although mammalian BZ CMs do not initiate proliferation endogenously like zebrafish BZ CMs, they do undergo drastic changes in terms of their transcriptome and chromosomal organization (van Duijvenboden et al., 2019. Circulation 140:864-879), which might potentiate their susceptibility to mitogenic stimuli.

Wu et al., 2019 (Wu et al. 2019. J Mol Cell Cardiol 128:160-178) describe a role for HMGA2 in pressure overload-induced cardiac remodeling. The authors showed that overexpression of HMGA2, followed by aortic banding, reduced myocardial hypertrophy in mice, as evidenced by a decreased cell surface area, decreased heart weight to body weight ratio and a reduced expression of hypertrophic markers. In contrast, knock down of HMGA2, followed by aortic banding, resulted in increased myocardial hypertrophy and increased heart weight to body weight ratio. In addition. Wu et al., 2020 (Wu et al., 2020. Cell Death Disease 160:1-13) reported that overexpression of HMGA1 in mice hearts increased apoptosis and exacerbated cardiac dysfunction. Neither of these documents show, or even suggest, that HMGA is involved in regulating cell division, let alone that upregulation of HMGA would promote cardiomyocyte proliferation.

However, we are still far from fully understanding the striking difference in regenerative capacity between zebrafish and mammalian hearts. Therefore, studying the molecular differences in the BZ between these species may lead to the identification of factors with the potential to stimulate mammalian heart regeneration.

### 2 BRIEF DESCRIPTION OF THE INVENTION

A screen was performed to identify transcriptional differences between a regenerating zebrafish heart and the non-regenerating mouse heart. This resulted in an extensive list of various genes and processes overlapping and diverging between the two species. This dataset forms a rich resource to identify novel drivers of cardiac regenerative capacity. The biological relevance of this comparison was functionally validated, resulting in the identification of a high mobility group A (HMGA) protein, especially Hmga1, as a key driver for cardiomyocyte (CM) proliferation. These results demonstrate that during zebrafish heart regeneration Hmga1a drives CM proliferation downstream of Nrg1 signaling by changing chromatin accessibility and induction of a profound gene expression program. In addition, forced Hmga1 expression in CM of an injured mouse heart promotes CM proliferation resulting in enhanced functional recovery.

The invention therefore provides a high mobility group A (HMGA) protein, for use in a method of promoting cardiomyocyte proliferation in an individual suffering from a structural cardiac muscle defect, comprising providing cardiomyocytes of at least part of the cardiac muscle of the individual with said HMGA protein, to thereby promote proliferation of said cardiomyocytes.

Said HMGA protein is preferably provided to said cardiomyocytes by systemic or local administration. Said HMGA protein is preferably provided by local administration, including by injection or infusion into the myocardium.

Said HMGA protein preferably is or comprises HMGA1, a part of HMGA1 comprising at least amino acid residues 21-89 of SEQ ID NO: 1, or a protein that is at least 75% identical to amino acid residues 21-89 of SEQ ID NO: 1 over the whole sequence.

In an embodiment, said HMGA protein is provided to said cardiomyocytes by an expression construct that expresses said HMGA protein in said cardiomyocytes. Said expression construct preferably is a viral vector, or a nucleic acid construct.

In an embodiment, said structural cardiac muscle defect is a congenital heart defect, such as a hypoplastic left heart syndrome or hypoplastic right heart syndrome.

In an embodiment, said structural cardiac muscle defect is in an adult who suffers from a myocardial infarction or heart failure.

The invention further provides an expression construct for functional expression of high mobility group A (HMGA) protein, preferably for functional expression of HMGA1, a part of HMGA1 comprising at least amino acid residues 21-89 of SEQ ID NO:1, or a protein that is at least 75% identical to amino acid residues 21-89 of SEQ ID NO: 1 over the whole sequence, in cardiomyocytes.

The invention further provides a pharmaceutical composition, comprising the expression construct according to the invention, and a pharmacologically acceptable excipient. Said expression construct preferably is a viral vector, such as an adenovirus-based vector or an adeno-associated virus (AAV)-based vector, or a nucleic acid construct, such as a mRNA-based construct.

The invention further provides a method of culturing cardiomyocytes in vitro, comprising providing cardiomyocytes with a HMGA protein or the expression construct according to the invention, and culturing said cardiomyocytes.

### 3 FIGURE LEGENDS

FIG. 1. (A) Tomo-seq reveals transcriptionally distinct regions in the injured mouse heart. Schematic overview of the workflow. (B) Transcriptional comparison between the zebrafish and mouse border zone identifies overlapping and divergent gene expression. Schematic overview of the workflow. (C) Scatterplot analysis comparing border zone expression as Log FC for homologous gene-pairs.

FIG. 2. hmga1a expression is correlated with regenerative potential. (A) qPCR analysis was done on cDNA libraries from whole mouse hearts at different postnatal time points. One-way ANOVA analysis indicates a significant difference in Hmga1 expression between different timepoints (p.val=0.0002). Dunnett's multiple comparison test was used to identify which specific columns significantly differ from the P1 timepoints. (B) Candidate genes were analysed with quantitative PCR on cDNA libraries obtained from isolated border zone (BZ) and remote myocardial (RM) tissue of injured mouse hearts 3, 7 or 14 days post myocardial infarction (induced by LAD occlusion). No differences between the BZ and RM was observed for Hmga1 (P=0.263). Foxp4 (P=0.134) and Igf2 (P=0.143). For Gpc1, a significant difference was observed where significantly higher expression is found in the RM compared to the BZ (P<0.001). P-values were obtained using two-way ANOVA testing. No significant interaction of zone and dpi were observed for any of the presented genes.

FIG. 3. 8 bp deletion causes frameshift in hmga1a coding region, leading to a strong reduction of hmga1a mRNA expression in the injury border zone. (A) Hmga1a protein structure, including 3 AT-hook DNA binding domains and an C-terminal acidic tail. (B) TALEN-based-8 bp deletion behind the start codon causing a frameshift leading to an early stop codon. (C.D) In situ hybridization against (C) hmga1a or (D) hmga1b in wild-type or hmga1a−/− hearts. n=3 for each condition. Dotted line indicates the injury area. Scale bars represent 100 μm.

FIG. 4. Zebrafish hmga1a is required for heart regeneration and allows border zone cardiomyocytes to assume specific cellular states. (A) Acid Fuchsin Orange G (AFOG) staining of hmga1a−/− and sibling hearts 30 dpi. Scale bars represent 100 μm. (B) Quantification of scar sizes. (C) Workflow of the isolation and sorting of nppa:mCitrine+ cardiomyocytes out of wild-type and hmga1a−/− hearts at 7 dpi. (D) Pseudo time analysis. One-dimensional SOM of z-score transformed expression profiles along the differentiation trajectory incurred by StemID analysis. Y-axis represents the eight modules with differentially expressed genes. X-axis represents the pseudo time in which the cells were ordered. (E) Distribution of genotype contribution across pseudotime. (F) Gene ontologies representing genes showing expression in specific modules. (G) In situ hybridization for hexokinase 1 (hk1) in the zebrafish border zone. Dotted line indicates the injury border. Scale bars indicate 50 μm. (H) Percentage of phosphorylated ribosomal S6 protein positive (pS6+) area relative to the tropomyosin+ area in a 300 μm wildtype and mutant border zone (left). Intensity of pS6 signal relative to underlying tropomyosin signal in wildtype and mutant border zone (right). (I) Immunofluorescent staining for tropomyosin (top. 1st row) and pS6 (2nd row). Myocardial pS6 signal (3rd row) was obtained after masking of the total pS6 for Tropomyosin. Scale bars indicate 200 μm.

FIG. 5. Zebrafish hmga1a is necessary for injury and NRG1 induced cardiomyocyte proliferation. (A) Quantification of proliferating border zone cardiomyocytes. (B) Quantification of proliferating cardiomyocytes in PBS or NRG1 injected zebrafish, either in hmga1a−/− or wild-type sibling hearts.

FIG. 6. Zebrafish hmga1a overexpression is sufficient to induce proliferation in cardiomyocytes. Quantification of proliferating cells. (A) total heart surface (myocardium+lumen). (B) the percentage of total heart surface covered with myocardium. (C) percentage of proliferating CMs. (D) the density of cardiomyocyte nuclei. (E) Quantification of myocardium covered surface area between control and Hmga1a-eGFP overexpressing hearts after >1 year.

FIG. 7. Hmga1 overexpression stimulates cell-cycle re-entry of mammalian cardiomyocytes and promotes functional recovery post-MI. (A) Workflow of Hmga1-eGFP overexpression in neonatal rat cardiomyocytes used in (B-D). (B-D) Proliferation marker quantification on eGFP only or Hmga1-eGFP transfected cells. For EdU, Ki67 and pHH3 quantification. 3 technical replicates were quantified per condition, except the pHH3 Hmga1-eGFP condition for which only 2 technical replicates were available. (E) Quantification of EdU+ cells within the border zone (BZ) or remote myocardium (RM) of hearts transfected with HA-Hmga1. At least 3 heart sections were quantified per heart. (F. H) The ejection fraction (F) or fractional shortening (H) was plotted against the average amount of transfected cells found in the BZ (defined as no further then 200 μm from the injury area). At least 3 heart sections were quantified per heart. Vertical grey line indicates the transfection efficiency cut-off used. (G. I) Quantification of the ejection fraction (G) or fractional shortening (I) of sham operated hearts, or MI hearts that were injected with either control virus or AAV9 (CMV: HA-Hmga1). Hearts were excluded that showed ineffective transfection (average of <30 transfected BZ cells).

FIG. 8. Hmga1 overexpression stimulates cell cycle re-entry of mouse cardiomyocytes and promotes functional recovery post-MI. Quantification of EdU+ (A) or Ki67+ (B) cells within the border zone (BZ) of hearts transduced with HA-Hmga1 or GFP. 3 heart sections were quantified per heart. Statistics were obtained using a one-way ANOVA followed by Tukey's multiple comparisons test. (C) Masson's trichrome staining of representative HA-Hmga1 and GFP transduced hearts at 42 dpi. Distance between sections is 400 um. Scale bars represent 1 mm. Scar quantification 42 dpi of the average angular scar size (D) or average % MI length/midline LV length (E) of hearts transduced with HA-Hmga1 or GFP. Statistics were obtained using unpaired t-tests. 42 dpi scar quantification of the average % MI length/midline LV length (D) of hearts transduced with HA-Hmga1 or GFP. Quantification of ejection fraction (E), fractional shortening (F), cardiac output (G) and stroke volume (H) at 42 dpi of sham and MI hearts transduced with HA-Hmga1 or GFP control virus. Statistics were obtained using a one-way ANOVA followed by Tukey's multiple comparisons test.

### 4 DETAILED DESCRIPTION OF THE INVENTION

### 4.1 Definitions

The term “high mobility group A (HMGA) protein”, as is used herein, refers to a chromatin-associated protein involved in the regulation of gene transcription. The protein preferentially binds to the minor groove of AT-rich regions in double-stranded DNA. Binding is mediated by the presence of three so called A-T hooks. The term HMGA includes reference to a functional variant comprising at least the central part of the protein, including the A-T hooks. There are two mammalian HMGA proteins. HMGA1 and HMGA2. A gene encoding HMGA1 resides on human chromosome 6p21.31, and is characterized by HUGO Gene Nomenclature Committee (HGNC) accession number 5010. NCBI Entrez Gene accession number 3159, and Ensembl accession number ENSG00000137309. The encoded protein is characterized by UniProt accession number P17096. A gene encoding HMGA2 resides on human chromosome 12q14.3, and is characterized by HGNC accession number 5009. NCBI Entrez Gene accession number 8091, and Ensembl accession number ENSG00000149948. The encoded protein is characterized by UniProt accession number P52926. A preferred HMGA protein is provided by HMGA1, a part of HMGA1 comprising at least amino acid residues 21-89 of SEQ ID NO: 1, or a protein that is at least 75% identical to at least amino acid residues 21-89 of SEQ ID NO: 1 over the whole sequence.

The term “structural cardiac muscle defect”, as is used herein, refers to a defect or disorder that is associated with the muscle cells, termed cardiomyocytes. A structural cardiac muscle defect is either congenital or develops later in life as a result of aging, injury, or infection. Examples include hypertrophic cardiomyopathy, hypoplastic heart syndrome and patent foramen ovale.

The term “myocardial infarction”, or heart attack, refers to a sudden ischemic death of myocardial tissue. Prolonged myocardial ischemia results in apoptosis and necrosis of cardiomyocytes in the infarcted heart. An adult mammalian heart hardly has regenerative capacity. Hence, an infarcted myocardium heals through fibrosis, i.e. the formation of a scar. Infarct healing is characterized by dilation, hypertrophy of viable segments, and progressive dysfunction.

The term “heart failure”, as is used herein, refers to a condition that develops when a heart is not able to pump enough blood, either because a heart can't fill up with enough blood, or because a heart is too weak to pump properly. Heart failure may be caused by a coronary heart disease, heart inflammation, high blood pressure, cardiomyopathy, or an irregular heartbeat.

The term “hypertrophic cardiomyopathy (HCM)”, as is used herein, refers to a thickening of the walls of the heart, normally of the left ventricle, that is associated with contractile dysfunction and potentially fatal arrhythmias. HCM is the most-common cause of sudden cardiac death in individuals younger than 35 years of age. HCM often precedes the development of heart failure.

The term “hypoplastic left heart syndrome”, as is used herein, refers to a range of congenital heart defects that affect normal blood flow through the heart. An underdeveloped and too small left ventricle is one of the symptoms of hypoplastic left heart syndrome.

The term “hypoplastic right heart syndrome”, as is used herein, refers to a range of congenital heart defects that affects normal blood flow through the heart. An absent, or underdeveloped and too small right ventricle is one of the symptoms of hypoplastic right heart syndrome.

### 4.2 Methods of Treatment

A method of treatment of an individual suffering from a structural cardiac muscle defect is aimed at inducing, at least in part, the proliferative capacity of cardiomyocytes. Methods of treatment involve means for increasing expression of a high mobility group A (HMGA) protein in cardiomyocytes. The provision of an increased expression level of a HMGA protein in cardiomyocytes of an individual include providing cardiomyocytes of the individual with said HMGA protein, or with a nucleic acid molecule encoding said HMGA protein. Said provision preferably is transient, meaning that said increased expression of a HMGA protein in temporally increased in cardiomyocytes, for example for a period of between 1 day and 6 months, such as between 1 week and 3 months.

It is thought that the provision of a HMGA protein to cardiomyocytes of at least part of the cardiac muscle of the individual will stimulate proliferation of said cardiomyocytes, especially of cardiomyocytes in the injury border zone. The increased presence of HMGA in these cells will promote chromatin reorganization leading to the induction of genes with a role in stress response, extracellular matrix production, metabolic reprogramming and cell proliferation.

The invention therefore provides a use of a high mobility group A (HMGA) protein, in the preparation of a medicament for promoting cardiomyocyte proliferation in an individual suffering from a structural cardiac muscle defect. The provision of cardiomyocytes of at least part of the cardiac muscle of the individual with said HMGA protein will promote proliferation of said cardiomyocytes.

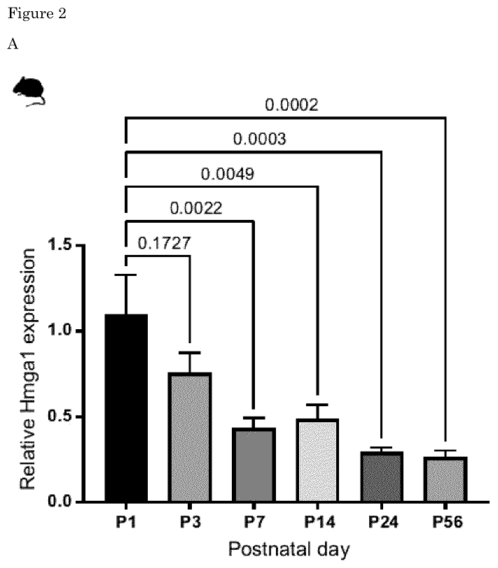

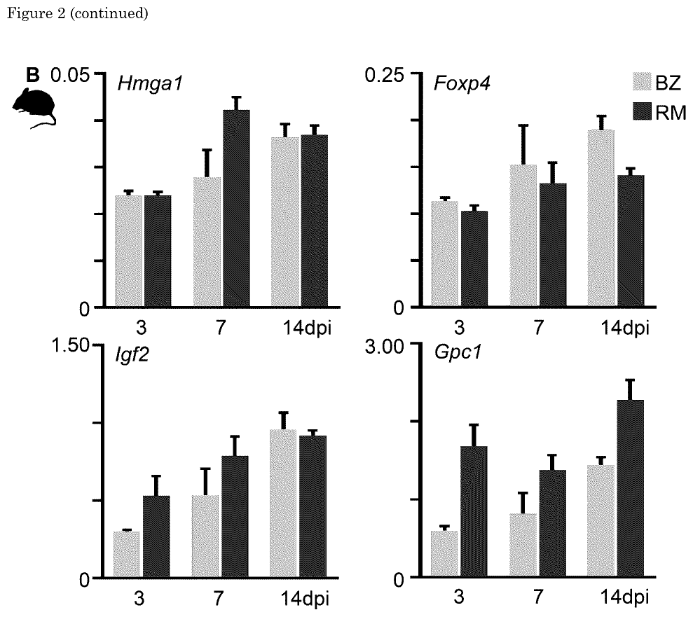

HMGA was identified in a screen for genes that are upregulated in the zebrafish border zone (BZ), but not in the mouse BZ. A total of 371 genes were identified (see Table 1). The 371 genes include Ensembl gene identifier ENSDARG00000033971, paired related homeobox 1 (prrx1), for which is has been showed that it is a key transcription factor that balances fibrosis and regeneration in the injured zebrafish heart (de Bakker et al., 2021. Development 148 (19): dev198937). For ENSDARG00000028335 (hmga1a), ENSDARG00000076120 (forkhead box p4; foxp4). ENSDARG00000033307 (insulin like growth factor 2: igf2) and ENSDARG00000090585 (glypican 1; gpc1), the BZ-enriched expression in zebrafish was confirmed through in situ hybridization and the lack of BZ enrichment in the mouse BZ using quantitative PCR analysis (FIG. 2B and data not shown). Of interest are genes ENSDARG00000076120 (foxp4). ENSDARG00000033307 (igf2), ENSDARG00000101482 (hexokinase 2; hk2), ENSDARG00000036096 (smad family member 3a; smad3a), ENSDARG00000034895 (transforming growth factor beta 1; tgfb1) and ENSDARG00000061508 (transforming growth factor beta receptor associated protein 1; tgfbrap1), especially ENSDARG00000076120 (foxp4) and ENSDARG00000101482 (hk2), which are thought to stimulate proliferation of cardiomyocytes, especially of cardiomyocytes in the injury border zone. The foxp4, igf2 and hk2 genes play a role in insulin growth factor signaling and the regulation of energy metabolism through regulation of glycolysis, which are required for CM proliferation in the injury border zone (Huang et al., 2013. PLoS One 8: e67266; Honkoop et al., 2019. Elife 8: e50163; Fukuda et al., 2020. EMBO Rep 21: e49752). The smad3a, tgfb1 and tgfbrap1 genes play a role in signal transduction through the TGF-beta growth factor pathway, which is an essential pathway for CM proliferation in the injury border zone (Chablais and Jazwinska, 2012. Development 139: 1921-1930; Peng et al., 2021. Front Cell Dev Biol 9: 632372). Overexpression of any one of the genes listed in Table 1, especially of ENSDARG00000076120 (foxp4), ENSDARG00000033307 (igf2), ENSDARG00000101482 (hk2), ENSDARG00000036096 (smad3a), ENSDARG00000034895 (tgfb1) and ENSDARG00000061508 (tgfbrap1), especially ENSDARG00000076120 (foxp4) and ENSDARG00000101482 (hk2), in cardiomyocytes of at least part of the cardiac muscle of an individual will stimulate proliferation of said cardiomyocytes, especially of cardiomyocytes in the injury border zone.

### 4.2.1 HMGA Protein Upregulation

The HMGA protein may be provided to induce proliferation of cardiomyocytes by systemic or local administration. Said HMGA protein preferably is expressed in a host cell. Commonly used expression systems for heterologous protein production include E. coli, baculovirus, yeast and mammalian cells. The efficiency of expression of recombinant proteins in heterologous systems depends on many factors, both on the transcriptional level and the translational level.

A HMGA protein may be produced in fungi, such as filamentous fungi or yeasts, including Saccharomyces cererisiae and Pichia pastoris.

A HMGA protein preferably is produced in mammalian cells, such as Chinese Hamster Ovary cells (CHO), human embryonic kidney (HEK) cells and derivatives thereof including HEK293 cells including HEK293T, HEK293E, HEK-293F and HEK-293FT (Creative Biolabs, NY, USA), and PER. C6 cells (Thermo Fisher Scientific, MA, USA).

Production of a HMGA protein is preferably produced by the provision of a nucleic acid molecule encoding said the HMGA protein to a cell of interest. Said nucleic acid, preferably DNA, is preferably produced by recombinant technologies, including the use of polymerases, restriction enzymes, and ligases, as is known to a skilled person. Alternatively, said nucleic acid is provided by artificial gene synthesis, for example by synthesis of partially or completely overlapping oligonucleotides, or by a combination of organic chemistry and recombinant technologies, as is known to the skilled person. Said nucleic acid is preferably codon-optimised to enhance expression of the HMGA protein in the selected cell or cell line. Further optimization preferably includes removal of cryptic splice sites, removal of cryptic polyA tails and/or removal of sequences that lead to unfavourable folding of the mRNA. The presence of an intron flanked by splice sites may encourage export from the nucleus. In addition, the nucleic acid preferably encodes a protein export signal for secretion of the HMGA protein out of the cell into the periplasm of prokaryotes or into the growth medium, allowing efficient purification of the HMGA protein.

Methods for purification of the HMGA protein are known in the art and are generally based on chromatography, such as ion exchange, to remove contaminants. In addition to contaminants, it may also be necessary to remove undesirable derivatives of the product itself such as degradation products and aggregates. Suitable purification process steps are provided in Berthold and Walter, 1994. Biologicals 22: 135-150.

As an alternative, or in addition, recombinant HMGA protein may be tagged with a specific tag by genetic engineering to allow the protein to attach to a column that is specific for said tag and therefore be isolated from impurities. The purified protein is then exchanged from the affinity column with a decoupling reagent. The method has been applied for purifying recombinant proteins. Conventional tags for proteins, such as histidine tag, are used with an affinity column that specifically captures the tag (e.g., a Ni-IDA column for histidine tag) to isolate the protein away from impurities. The protein is then exchanged from the column using a decoupling reagent according to the specific tag (e.g., imidazole for histidine tag). This method is more specific, when compared with traditional purification methods. Suitable further tags include c-myc domain, hemagglutinin tag, glutathione-S-transferase, maltose-binding protein, FLAG tag peptide, biotin acceptor peptide, streptavidin-binding peptide and calmodulin-binding peptide, as presented in Chatterjee, 2006. Cur Opin Biotech 17: 353-358). Methods for employing these tags are known in the art and may be used for purifying the HMGA protein.

Said HMGA protein may be provided by a cell penetrating peptide to promote delivery of said protein into cardiomyocytes. For this, said HMGA protein may be fused to a peptide of 3-50 amino acids, preferably 5-20 amino acids such as 6-12 amino acids, that comprises positively charged amino acids such as ornithine, lysine or arginine, comprises sequences that contain an alternating pattern of polar, charged amino acids and non-polar, hydrophobic amino acids, or comprises only apolar amino acid residues. Suitable peptides include a part of human immunodeficiency virus 1 tat, Herpes Simplex Virus 1 tegument protein VP22, Drosophila antennapedia, a poly-arginine peptide, poly-methionine peptide, a poly-glycine peptide, a cyclic poly-arginine, a peptide with the amino acid sequence RMRRMRRMRR, and variants or combinations thereof.

Said HMGA protein, preferably purified HMGA protein, may be provided by systemic or local administration, preferably by local administration. Said HMGA protein may be provided by injection or infusion into the myocardium, for example by employing a catheter. Said injection or infusion may be accomplished by use of external pump or of a fully implantable device. Said external pump is preferably equipped with a percutaneous catheter, tunneled or not tunneled, or equipped with a subcutaneous injection port and an implanted catheter.

As an alternative, said HMGA protein, preferably purified HMGA protein, is provided into a coronary artery that supplies the region comprising the structural cardiac muscle defect with blood.

Similarly, administration of a protein product of any one of the genes listed in Table 1, especially of ENSDARG00000076120 (foxp4), ENSDARG00000033307 (igf2), ENSDARG00000101482 (hk2), ENSDARG00000036096 (smad3a), ENSDARG00000034895 (tgfb1) and ENSDARG00000061508 (tgfbrap1), especially ENSDARG00000076120 (foxp4) and ENSDARG00000101482 (hk2), that is produced in an expression system, may be provided by systemic or local administration, to cardiomyocytes in order to stimulate proliferation of said cardiomyocytes, especially of cardiomyocytes in the injury border zone. Said protein may be tagged and/or provided with a cell penetrating peptide.

### 4.2.2 Expression of HMGA Protein

As an alternative, expression of HMGA in a cardiomyocyte may be provided by an expression construct for functional expression of HMGA in cardiomyocytes. Said expression construct preferably enables functional expression of HMGA in cardiomyocytes, preferably specific expression of HMGA in cardiomyocytes. Said cardiomyocyte-specific expression may be provided, for example, by expressing HMGA under control of a cardiomyocyte-specific promoter, such as the cardiac troponin T (Tnnt2) promoter (Wu et al., 2010. Genesis 48: 63-72), the cardiomyocyte-specific Na(+)-Ca(2+) exchange promoter (Agostini et al., 2013. Biomed Res Int 2013: 845816), or the cardiac myosin light chain 2 promoter (Griscelli et al., 1997. C R Acad Sci III 320: 103-112).

Said expression construct may be provided as a nucleic acid molecule, or provided by a vector, especially a viral vector, to deliver the nucleic acid molecule into cardiomyocytes of the individual. Said viral vector preferably provides temporal expression of the nucleic acid molecule. Said viral vector preferably is a recombinant adenovirus-based vector, an adenovirus associated virus-based vector, an alphavirus-based vector, a herpes simplex virus-based vector, or a pox virus-based vector. Said viral vector most preferably is a adenoviral-based vector or a self-amplifying alphavirus-based replicon vector (Ljungberg and Liljeström, 2015. Expert Rev Vaccines 14: 177-194).

Packaging of a viral vector in a viral particle, or viral-like particle, is known in the art, including transfection of packaging cells that express structural and packaging genes.

Methods for delivery of a viral vector that transduces HMGA to a cardiomyocyte include administration by a parenteral route, such as subcutaneous, intradermal, intramuscular, intravenous, intralymphatic, and intranodal administration. Said viral vector that transduces HMGA is preferably provided by local administration, for example into the myocardium, or into a coronary artery that supplies the region comprising the structural cardiac muscle defect with blood.

Said nucleic acid molecule may also be provided as a DNA molecule that expresses said HMGA upon delivery to a cardiomyocyte. Said DNA molecule may comprise modified nucleotides, for example to increase half live of the molecule. For example, said nucleic acid molecule may be provided in a plasmid, or as linear DNA. Non-virus mediated delivery of a DNA molecule according to the invention include lipofection, microinjection, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™, and SAINT™). Cationic and neutral lipids that are suitable for efficient lipofection of polynucleotides include those of WO 91/17424 and WO 91/16024. Delivery can be to cells (e.g. in vitro or ex vivo administration) or to target tissues (e.g. in vivo administration. Said DNA molecule may also be packaged, for example in lipid vesicles such as a virosome, a liposome, or immunoliposome, prior to delivery of said DNA molecule to an individual in need thereof.

Said nucleic acid molecule may also be provided as a RNA molecule that expresses HMGA upon delivery to a cardiomyocyte. Said RNA molecule may be synthesized in vitro, for example by a DNA dependent RNA polymerase such as T7 polymerase, T3 polymerase, SP6 polymerase, or a variant thereof. Such variant may include for instance a mutant T7 RNA polymerase that is capable of utilizing both canonical and non-canonical ribonucleotides and deoxynucleotides as substrates (Kostyuk et al., 1995. FEBS Lett. 369: 165-168; Padilla et al., 2002. Nucl. Acids Res. 30(24): e138), or a RNA polymerase variant displaying higher thermostability such as Hi-T7™ RNA Polymerase from New England Biolabs (Boulain et al., 2013. Protein Eng Des Sel. 26(11): 725-734).

Said RNA molecule may encompass, for example, a synthetic cap analogue (Stepinski et al., 2001. RNA 7: 1486-1495), one or more regulatory elements in the 5′-untranslated region (UTR) and/or the 3′-UTR that stabilize said RNA molecule and/or increases protein translation (Ross and Sullivan, 1985. Blood 66: 1149-1154), and/or modified nucleosides to increase stability and/or translation (Kariko et al., 2008. Mol Ther 16: 1833-1840), and/or to decrease an inflammatory response (Kariko et al., 2005. Immunity 23: 165-175). (2005). In addition, said RNA molecule preferably encompasses a poly(A) tail to stabilize the RNA molecule and/or to increase protein translation (Gallie, 1991. Genes Dev 5: 2108-2116).

Said nucleic acid molecule may be delivered to an individual in the presence or absence of a carrier. Said carrier preferably allows prolonged expression in vivo of HMGA protein in a cardiomyocyte. Said carrier may be one or more of a cationic protein such as protamine, a protamine liposome, a polysaccharide, a cation, a cationic polymer, a cationic lipid, cholesterol, polyethylene glycol, and a dendrimer. For example, said RNA molecule may be delivered as a naked RNA molecule, complexed with protamine, associated with a positively charged oil-in-water cationic nanoemulsion, associated with a chemically modified dendrimer and complexed with polyethylene glycol (PEG)-lipid, complexed with protamine in a PEG-lipid nanoparticle, associated with a cationic polymer such as polyethylenimine, associated with a cationic polymer such as PEI and a lipid component, or associated with a polysaccharide such as, for example, chitosan, in a cationic lipid nanoparticle such as, for example, 1,2-dioleoyloxy-3-trimethylammoniumpropane (DOTAP) or dioleoylphosphatidylethanolamine (DOPE) lipids), complexed with cationic lipids and cholesterol, and complexed with cationic lipids, cholesterol and PEG-lipid, as is described in Pardi et al., 2018. Nature Reviews 17: 261-279).

Methods to introduce a nucleic acid into a cell include lipofection, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™, and SAINT™). Cationic and neutral lipids that are suitable for efficient receptor-recognition lipofection of polynucleotides include those of WO 91/17424 and WO 91/16024. Delivery to a target tissue preferably is by systemic or, preferably, local administration to a region comprising cardiomyocytes.

Said nucleic acid molecule that expresses HMGA upon delivery to a cardiomyocyte may be administered by a parenteral route, including subcutaneous, intradermal, intramuscular, intravenous, intralymphatic, intranodal administration. As is known to a person skilled in the art, a carrier may be selected that is suited for a specific mode of administration in order to achieve a desirable outcome.

Similarly, administration of a nucleic acid molecule that encodes a protein product of any one of the genes listed in Table 1, especially of ENSDARG00000076120 (foxp4), ENSDARG00000033307 (igf2), ENSDARG00000101482 (hk2), ENSDARG00000036096 (smad3a), ENSDARG00000034895 (tgfb1) and ENSDARG00000061508 (tgfbrap1), especially ENSDARG00000076120 (foxp4) and ENSDARG00000101482 (hk2), may be provided by systemic or local administration, to have it expressed in cardiomyocytes in order to stimulate proliferation of said cardiomyocytes, especially of cardiomyocytes in the injury border zone.

### 4.2.3 Pharmaceutical Composition

Further provided is a pharmaceutical composition comprising HMGA protein or comprising an expression construct for functional expression of high mobility group A (HMGA) protein, preferably for functional expression of HMGA1, a part of HMGA1 comprising at least amino acid residues 21-89 of SEQ ID NO:1, or a protein that is at least 75% identical to amino acid residues 21-89 of SEQ ID NO:1 over the whole sequence, in cardiomyocytes, and a pharmacologically acceptable excipient. Said pharmaceutical composition preferably is a sterile isotonic solution.

Said pharmaceutically acceptable excipient preferably is selected from diluents, binders or granulating ingredients, a carbohydrate such as starch, a starch derivative such as starch acetate and/or maltodextrin, a polyol such as xylitol, sorbitol and/or mannitol, lactose such as α-lactose monohydrate, anhydrous α-lactose, anhydrous β-lactose, spray-dried lactose, and/or agglomerated lactose, sugars such as dextrose, maltose, dextrate and/or inulin, glidants (flow aids) and lubricants, and combinations thereof.

A pharmaceutical composition comprising HMGA preferably comprises an excipient to maintain protein stability, solubility, and pharmaceutical acceptance. Said excipient preferably is selected from, but not limited to, urea, L-histidine, L-threonine, L-asparagine, L-serine, L-glutamine, polysorbate, polyethylene glycol, propylene glycol, polypropylene glycol, or a combination of two or more of the above. Salts and buffers are known to effect protein stability, especially in frozen solutions and freeze-dried solids because of the increased concentrations and possible pH changes. In addition, various sugars may protect the conformation of proteins in aqueous solutions and during freeze-drying. For example, nonreducing disaccharides such as sucrose and trehalose are potent and useful excipients to protect protein conformation in aqueous solutions and freeze-dried solids, whereas reducing sugars such as maltose and lactose can degrade proteins during storage. Said excipients may further include sugar alcohols such as inositol, and/or amino acids such as arginine may protect protein conformation against dehydration stresses. Further excipients may include a surfactant, such as a nonionic surfactant, and/or a polymer such as hydroxyethyl starch.

Said expression construct preferably mediates specific expression of HMGA in cardiomyocytes, for example by expressing HMGA under control of a cardiomyocyte-specific promoter.

Said expression construct is either a nucleic acid molecule, such as a DNA or RNA molecule, or a viral vector. Said viral vector preferably provides temporal expression of the nucleic acid molecule in a cardiomyocyte. Said viral vector preferably is a recombinant adenovirus-based vector, an adenovirus associated virus-based vector, an alphavirus-based vector, a herpes simplex virus-based vector, or a pox virus-based vector. Said viral vector most preferably is a adenovirus-based vector, a self-amplifying alphavirus-based replicon vector (Ljungberg and Liljeström, 2015. Expert Rev Vaccines 14: 177-194), or an adeno-associated virus (AAV)-based vector.

As an alternative, said expression construct is a mRNA-based expression construct. As is indicated herein above, said mRNA-based expression construct or RNA molecule may encompass, for example, a synthetic cap analogue (Stepinski et al., 2001. RNA 7: 1486-1495), one or more regulatory elements in the 5′-untranslated region (UTR) and/or the 3′-UTR that stabilize said RNA molecule and/or increases protein translation (Ross and Sullivan, 1985. Blood 66: 1149-1154), and/or modified nucleosides to increase stability and/or translation (Kariko et al., 2008. Mol Ther 16: 1833-1840), and/or to decrease an inflammatory response (Kariko et al., 2005. Immunity 23: 165-175). In addition, said RNA molecule preferably encompasses a poly(A) tail to stabilize the RNA molecule and/or to increase protein translation (Gallie, 1991. Genes Dev 5: 2108-2116).

Excipients for a nucleic acid molecule as an expression construct, such as a mRNA-based expression construct, include lipids, lipid-like compounds, and lipid derivatives, especially cationic or ionizable lipid materials, polymeric materials, including polyamines, dendrimers, and copolymers, especially cationic polymers such as polyethylenimine and/or polyamidoamine, and peptides such as protamine.

A pharmaceutical composition comprising a naked nucleic acid molecule as an expression construct, such as a naked mRNA expression molecule, preferably comprises excipients such Ringer's solution and Ringer's lactate, as is known to a person skilled in the art.

Similarly, said pharmaceutical composition comprising may comprise a protein product of any one of the genes listed in Table 1, especially of ENSDARG00000076120 (foxp4), ENSDARG00000033307 (igf2), ENSDARG00000101482 (hk2), ENSDARG00000036096 (smad3a), ENSDARG00000034895 (tgfb1) and ENSDARG00000061508 (tgfbrap1), especially ENSDARG00000076120 (foxp4) and ENSDARG00000101482 (hk2), or an expression construct for expression of a nucleic acid molecule that encodes said protein product.

### 5 EXAMPLES

### Example 1

### Materials and Methods

### Animal Experiments

All procedures involving animals were approved by the local animal experiments committees and performed in compliance with animal welfare laws, guidelines, and policies, according to national and European law.

### Zebrafish and Mouse Lines

The following zebrafish lines were used: TL, TgBAC(nppa:mCitrine) (Honkoop et al., 2019. ELife 8: 1-27), Tg(myl7:CreER)pd10 (Kikuchi et al., 2010. Nature 464: 601-605), Tg(myl7:DsRed2-NLS) (Mably et al., 2003. Curr Biol 13: 2138-2147). The hmga1a−/− was produced using a TALEN based strategy, targeting the region adjacent to the transcription start site, leading to a frameshift and an early stop codon (data not shown). The tg(ubi:Loxp-stop-Loxp-hmga1a-eGFP) was produced using gBlocks and Gibson Assembly, using the pDESTp3A destination vector (Kwan et al., 2007. Dev Dyn 236: 3088-3099) and the p5E ubi promotor (Mosimann et al., 2011. Development 138: 169-177). The following mouse lines were used: C57BL/6J males (Charles River), tg(Nppb:katushka) (Sergeeva et al., 2014. Cardiovas Res 101: 78-86).

### Myocardial Infarction in Mice

Cardiac ischemic injuries were accomplished by permanent occlusion of the left anterior descending artery (LAD), previously described in (Sergeeva et al., 2014. Cardiovas Res 101: 78-86), using adult male mice between 7-12 weeks of age.

### Transthoracic Echocardiography

Transthoracic echocardiography was performed to address heart function. In brief, mice were anesthetized with a mixture of ketamine and xylazine by IP injection, and hair was shaved from the thorax. A tracheal tube two-dimensional transthoracic echocardiography on sedated, adult mice (2% isoflurane) using a Visual Sonic Ultrasound system with a 30 MHz transducer (VisualSonics Inc., Toronto, Canada). The heart was imaged in a parasternal longaxis as well as short-axis view at the level of the papillary muscles, to record Mmode measurements, determine heart rate, wall thickness, and end-diastolic and end-systolic dimensions. Fractional shortening (defined as the end-diastolic dimension minus the end-systolic dimension normalized for the end-diastolic dimension) as well as ejection fraction (defined as the stroke volume normalized for the end-diastolic volume), were used as an index of cardiac contractile function.

Virus-mediated overexpression of Hmga1 in neonatal rat cardiomyocytes Cardiomyocytes of 1-day-old neonatal rat hearts were isolated by enzymatic dissociation with trypsin (Thermo Fisher Scientific, #15400054) and cultured as described in Gladka et al., 2021 (Gladka et al., 2021. Nature Comm 12: 84). Overexpression of Hmga1 was accomplished through lenti-virus mediated delivery of pHAGE2-EF1a:Hmga1-T2A-GFP construct.

### Virus Injections in Mice

To induce Hmga1 expression, we employed AAV9 virus which preferentially targets CMs, carrying a CMV:HA-Hmga1 cassette. Upon myocardial infarction, hearts were injected twice with 15 μl virus (1×10\hat{ }11 virus particles/mouse) in regions bordering the area at risk of ischemic injury.

### 5-Ethynyl-2′-Deoxyuridine (EdU) Injections in Mice

To assess cell-cycle re-entry at 14 days post MI, adult mice received bi-daily intraperitoneal injections starting at day 2 (resulting in 6 EdU injections in total, per mice). EdU concentrations were determined based on the individual weight of each mouse (50 pg/g).

### In Situ Hybridization

Paraffin sections: After o/n fixation in 4% PFA, hearts were washed in PBS twice, dehydrated in EtOH, and embedded in paraffin. Serial sections were made at 10 μm thickness. In situ hybridization was performed on paraffin-sections as previously described (Moorman et al., 2001. J Histochem Cytochem 49: 1-8) except that the hybridization buffer used did not contain heparin and yeast total RNA. Cryo-sections were obtained as described earlier. In situ hybridization was performed as for paraffin, however sections were prefixed for 10 min in 4% PFA+0.25% glutaraldehyde before Proteinase K treatment. Moreover, slides were fixed for 1 hr in 4% PFA directly after staining.

qPCR

Total RNA was isolated from heart ventricles using TRIzol reagent reagent (Life Technologies, Carlsbad, CA, USA) according to the manufacturer's instructions. Total RNA (1 pg) was reverse transcribed using iScript cDNA Synthesis Kit (Bio-Rad, Hercules, CA, USA). Real-time PCR was performed using iQ SYBRgreen kit and CFX96 real-time PCR detection system (BioRAD). Data was normalized using housekeeping genes Gapdh or Hprt and Eefele.

### (Immuno)histochemistry

Adult zebrafish ventricles were isolated and fixed in 4% PFA (4° C. O/N on a shaker). The next day, the hearts were washed 3×10 minutes in 4% sucrose phosphate buffer, after which they were incubated at RT for at least 5 h in 30% sucrose phosphate buffer until the hearts floated. Then, they were embedded in cryo-medium (OCT). Cryo-sectioning of the hearts was performed at 10 micron thickness. Primary antibodies used include anti-PCNA (Dako #M0879, 1:800), anti-GFP (Aves Labs #GFP-1010, 1:1000), and anti-Mef2c (Santa Cruz #SC313, Biorbyt #orb256682 both 1:1000). Antigen retrieval was performed by heating slides containing heart sections at 85° C. in 10 mM sodium citrate buffer (pH 6) for 15 min. On mouse paraffin sections: After o/n fixation in 4% PFA, hearts were washed in PBS twice, dehydrated in EtOH and Xylene and embedded in paraffin. Serial sections were made at 6 μm. Antigen retrieval was performed by heating slides containing heart sections under pressure at 120° C. in 10 mM sodium citrate buffer (pH 6) for 1 hour. Primary antibodies used were anti-HMGA1 (Santa Cruz #sc393213), anti-HA (Abcam #ab9111) and anti-PCM (Sigma #HPA023370). EdU was visualized with the Click-iT EdU Cell Proliferation Imaging Kit, Alexa Fluor 647, ThermoFisher, #C10340, according to the instructions. For both mouse and zebrafish tissue, Secondary antibodies include Anti-chicken Alexa488 (Thermofisher, #A21133, 1:500), anti-rabbit Alexa555 (Thermofisher, #A21127, 1:500), anti-mouse Cy5 (Jackson ImmunoR, #118090, 1:500) used at a dilution of 1:500. DAB staining was performed using the DAB Substrate Kit (SK-4100) (Vector Laboratories). Nuclei were shown by DAPI (4′,6-diamidino-2-phenylindole) staining. Images of immunofluorescence staining are single optical planes acquired with a Leica Sp8 microscope.

### Quantification and Statistics

All data was quantified in a double-blinded fashion. Unless stated otherwise, statistical testing was performed by unpaired T-tests. Histological quantifications of border-zone specific populations was performed on cardiomyocytes situated within 150 μm from the wound border on 3 sections per heart, of at least 3 hearts. qPCR results comparing Hmga1 expression throughout postnatal development (FIG. 2) was statistically tested using one-way ANOVA and Dunnett's multiple comparison test. qPCR results comparing target gene expression at different time-points was statistically tested using two-way ANOVA.

### Zebrafish Intraperitoneal Injections of NRG1

Intraperitoneal injections of human recombinant NRG1 (Peprotech: recombinant human heregulin-b1, catalog #:100-03) were performed as described by Kinkel et al. (Kinkel et al., 2010. J Vis Exp 42: e2126). Fish were sedated using MS222 (0.032% wt/vol). Injections were performed using a Hamilton syringe (Gauge 30), cleaned before use by washing in 70% ethanol followed by 2 washes in PBS. Injection volumes were adjusted on the weight of the fish (30 μl/g) and a single injection contained 60 μg/kg of human recombinant NRG1 (diluted in PBS/BSA 0.1%).

### Tomo-Sequencing

Under a fluorescent stereoscope, injured mouse hearts were isolated and cut open longitudinally and tissue was selected based on the katushka signal. Tissue was isolated from the injured hearts (n=3) containing part of the injury, katushka signal and part of the remote myocardium respectively. Tomo-seq was conducted as previously described (Junker et al., 2014. Cell 159: 662-675) whereby the tissue was embedded in Jung tissue freezing medium (Leica), sectioned at 20 μm from the injured area to the remote myocardium, of which every fifth section was collected into a single tube. RNA was extracted from each tube using Trizol (Ambion) after adding a defined amount of spike-in RNA to correct for technical variations during the downstream processing. RNA-seq was performed as previously described (Junker et al., 2014. Cell 159: 662-675) including the reverse transcription using primers containing a tube-specific barcode. The barcoding of the cDNA allowed for the pooling of material, since the barcode could later be used to determine the positional origin of the labeled transcript. The pooling of cDNA was followed by linear amplification and sequencing.

### Single Cell RNA Sequencing

Tg(nppa:mCitrine) positive cells showing high mCitrine expression were isolated from cyoinjured zebrafish hearts (7 days post injury). From 12 hmga1a−/− mutant hearts, 768 cells were isolated. From 12 hmga1a+/+ wild-type hearts, 768 cells were isolated. Single-cell sequencing libraries were prepared using SORT-seq (Muraro et al., 2016. Cell Systems 3: 385-394). Live cells were sorted into 384-well plates with Vapor-Lock oil containing a droplet with barcoded primers, spike-in RNA and dNTPs, followed by heat-induced cell lysis and cDNA syntheses using a robotic liquid handler. Primers consisted of a 24 bp polyT stretch, a 4 bp random molecular barcode (UMI), a cell-specific 8 bp barcode, the 5′ Illumina TruSeq small RNA kit adapter and a T7 promoter. After cell-lysis for 5 min at 65° C., RT and second strand mixes were distributed with the Nanodrop II liquid handling platform (Innovadyne). After pooling all cells in one library, the aqueous phase was separated from the oil phase, followed by IVT transcription. The CEL-Seq2 protocol was used for library prep (Hashimshony et al., 2016. Genome Biol 17: 1-7). Illumina sequencing libraries were prepared with the TruSeq small RNA primers (Illumina) and paired-end sequenced at 75 bp read length on the Illumina NextSeq platform.

### ATAC- and RNA-Sequencing

Nuclei isolation was performed on whole zebrafish hearts 14 days post tamoxifen treatment. Fish either contained 3 transgenes allowing cardiomyocyte specific overexpression of hmga1a-eGFP tg(ubi:Loxp-stop-Loxp-hmga1a-eGFP); Tg(myl7:DsRed2-NLS); Tg(myl7:CreER)pd10 or formed the control fish containing only two transgenes (Tg(myl7:DsRed2-NLS); Tg(myl7:CreER)pd10). A total of 50,000 DsRed positive control nuclei were sorted as well as 45,000 DsRed/GFP+ hmga1a nuclei, both originating from 20 hearts each. DNA was isolated, treated with transposase TDE1 (Nextera Tn5 Transposase from Nextera kit; Illumina, cat. no. FC-121-1030) as described previously in Buenrostro et al., 2013. (Buenrostro et al., 2013. Nat Methods 10: 1213-1218). Illumina sequencing libraries were generated and sequenced using the NextSeq platform. Bulk RNA-sequencing was performed on whole zebrafish ventricles 14 days post tamoxifen treatment. Fish either contained 2 transgenes allowing cardiomyocyte specific overexpression of hmga1a-eGFP tg(ubi:Loxp-stop-Loxp-hmga1a-eGFP); Tg(myl7:CreER)pd10 or formed the control fish containing only one transgenes (Tg(myl7:CreER)pd10). RNA was isolated using Trizol (Ambion). Library preparation was performed following the CEL-Seq1 protocol described in (Junker et al., 2014. Cell 159: 662-675). Again, Illumina sequencing libraries were generated and sequenced using the NextSeq platform.

### Bioinformatical Analysis: Tomo-Sequencing Data

Mapping was performed against the zebrafish reference assembly version 9 (Zv9) and the mouse reference assembly version 9 (mm9). The Tomo-Sequencing analysis was done based on the log 2-transformed fold-change (zlfc) of the Z score (number of standard deviations above the mean) of all genes. Bioinformatic analyses were largely performed with R software using custom-written code (R Core Team, 2013). Hierarchical cluster analysis on the entire dataset (after Z score transformation) was performed on all genes with a peak in >4 consecutive sections (Z score>1). Based on hierarchical clustering analysis, together with maker gene expression (BZ markers Nppa, Nppb, Des), we defined the locations of the injury area (IA), border zone (BZ) and remote myocardium (RM) within our datasets. To transcriptionally compare the zebrafish and mouse border zone, we first pooled all injury area, border zone and remote zone regions from the different timepoints into one species specific dataset per species, resulting in 14 (IA), 43 (BZ) and 43 (RM) sections in the zebrafish dataset and 14 (IA), 65 (BZ) and 54 (RM) sections in the mouse dataset. Gene ontology analysis was performed on these combined lists using the R package edgeR (Robinson et al., 2009. Bioinformatics 26: 139-140). These gene lists were subjected to GO analysis using the online tool DAVID (Huang et al., 2009. Nature Protocols 4: 44-57). The transcriptional comparison between the zebrafish and mouse border zone was performed by scatter plotting annotated gene-pairs annotated in Ensembl(v89). Genes with no annotated homolog were excluded from analysis. Genes with multiple annotated homologs were plotted as separate gene pairs. Next, gene pairs were selected using the following tresholds; Upregulated in both the mouse and zebrafish BZ: Zebrafish log FC>0.5, P.val<0.05; Mouse: log FC>0.5, P.val<0.05. Downregulated in both the mouse and zebrafish BZ: Zebrafish: log FC<−0.5, P.val<0.05; Mouse: log FC<−0.5, P.val<0.05. Upregulated in the zebrafish BZ, but not the mouse BZ: Zebrafish: log FC>0.5, P.val<0.05; Mouse: log FC<0. Upregulated in the mouse BZ, but not the zebrafish BZ: Zebrafish: log FC<0; Mouse: log FC>0.5, P.val<0.05. Furthermore, after determining the zebrafish and mouse specific gene-pairs, gene-pairs were removed of which at least one paralogous gene showed expression outside of the selection thresholds, accounting for redundant functions between paralogous genes. These gene-pairs were subjected to GO analysis using their mouse name in the online tool DAVID (Huang et al., 2009. Nature Protocols 4: 44-57).

### Bioinformatical Analysis: Single-Cell RNA Sequencing Data

In total, eight 384-well plates were sequenced, containing one cell per well. Four plates were obtained per genotype (hmga1a−/− or wild-type). Sequencing one of the wild-type containing plates failed, hence no data was obtained. Mapping was performed against the zebrafish reference assembly version 9 (Zv9). Based on the distribution of the log 10 total reads plotted against the frequency, we introduced a cutoff at minimally 600 reads per cell before further analysis, reducing the amount of analysed cells to 653 wild-type and 658 hmga1a−/− cells (1311 cells in total). Batch-effects were analyzed and showed no plate-specific clustering of certain clusters. Next, the single cell RNA sequencing data was analyzed using an updated version (RaceID2) of the previously published RaceID algorithm (Grun et al., 2015. Nature 525: 251-255), resulting in the characterization of 6 main cell clusters with transcriptionally distinct characteristics. To identify modules of co-expressed genes along a specific differentiation trajectory (defined as a succession of significant links between clusters as identified by StemID, as previously published (Grun et al., 2016. Cell Stem Cell 19: 266-277) all cells assigned to these links were assembled in pseudo-temporal order based on their projection coordinate. Next, all genes that are not present with at least two transcripts in at least a single cell are discarded from the sub-sequent analysis. Next, a local regression of the z-transformed expression profile for each gene is computed along the differentiation trajectory. These pseudo-temporal gene expression profiles are topologically ordered by computing a one-dimensional self-organizing map (SOM) with 1000 nodes. Due to the large number of nodes relative to the number of clustered profiles, similar profiles are assigned to the same node. Only nodes with more than three assigned profiles are retained for visualization of co-expressed gene modules. Neighboring nodes with average profiles exhibiting a Pearson's correlation coefficient >0.9 are merged to common gene expression modules. These modules are depicted in the final pseudo-temporal map. Analyses were performed as previously published (Grun et al., 2016. Cell Stem Cell 19: 266-277).

Bioinformatical analysis: ATAC- and RNA-sequencing data Both ATAC- and RNA-seq data was mapped against the zebrafish reference assembly version 10 (DanRer10). The ATAC-sequencing data were uploaded to the Galaxy web platform, and we used the public server at usegalaxy.org to analyze the data (Afgan et al., 2016. Nucleic Acids Res 44: W3-W10). Mapping of the ATAC-sequencing data was done with Bowtie2 (Langmead and Salzberg, 2012. Nature Methods 9: 357-959). Peaks were called using MACS2 peak calling, using a minimum q-value of 0.05 (minimum FDR) cutoff to call significant regions. Q-values were calculated from p-values using Benjamini-Hochberg procedure (--qvalue). The genomic distribution of accessible regions was defined using annotations of Ensembl BioMart, DanRer10 build. For promoter accessibility analysis the ATAC-seq signal was distributed into promoter bins defined as 1500 bp upstream and 500 bp of the canonical TSS (BioMart, DanRer10 build). Motif enrichment analysis was performed using the web based MEME suite tool Analysis of Motif Enrichment (AME v5.3.0) (Bailey et al., 2009. Nucleic Acids Res 43: W39-W49; McLeay & Bailey, 2010. BMC Bioinformatics. 11: 165). Differentially expressed genes were obtained from the RNA-sequencing data using the R package edgeR (Robinson et al., 2009. Bioinformatics 26: 139-140). These gene lists were subjected to GO analysis using the online tool DAVID (Huang et al., 2009. Nature Protocols 4: 44-57).

### Results

### Comparison of Transcriptional Border Zone Profiles Reveals Species Specific Gene Expression

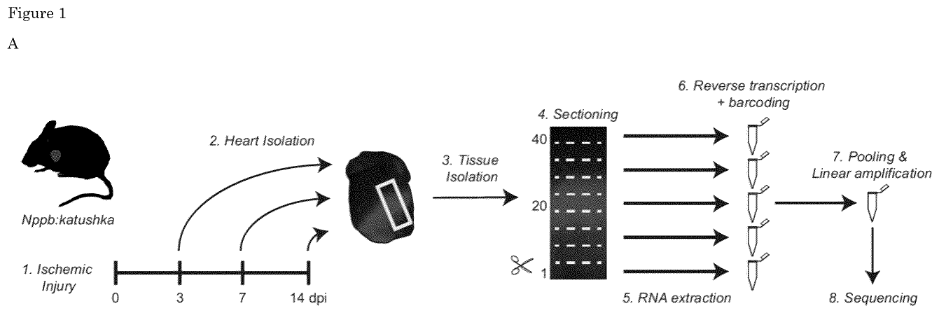

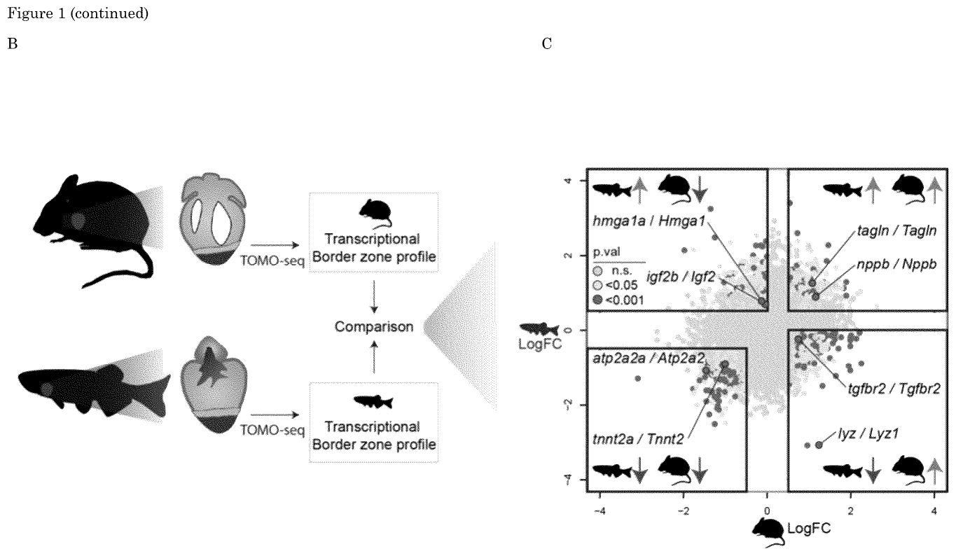

We previously used spatially resolved transcriptomics (TOMO-seq) to determine gene expression in the zebrafish BZ at 3, 7 and 14 days post injury (dpi) (Junker et al., 2014. Cell 159: 662-675; Wu et al., 2015. Develop Cell 36: 36-49). To allow comparison between the zebrafish and mouse BZ, we performed similar TOMO-seq experiments on injured mouse hearts. Mice were subjected to myocardial infarction (MI) through permanent occlusion of the left-anterior descending coronary artery. Spatial patterns of Nppb/Katushka in the BAC-Nppb-Katushka mice allowed for the localization of the BZ (Sergeeva et al., 2014) and the BZ with the surrounding injury area and remote myocardium were isolated at 3, 7 and 14 days post-MI (FIG. 1A). Next, the isolated tissue was cryosectioned from injury area to remote area and each section was subjected to RNA-sequencing (RNA-seq). Pearson's correlation analysis across all genes for each pairwise combination of sections revealed clusters of genes with expression in distinct areas. Based on these gene clusters as well as marker gene expression, we identified the locations of the injury area (cluster 1, Rhoc, Fstl1, Tmsb4x), border zone (Cluster 2, Nppa, Des, Ankrd1) and remote myocardium (cluster 3, Tnnt2, Tnni3, Ech1) within the TOMO-seq datasets (Lacraz et al., 2017. Circulation 136: 1396-1409; van Duijvenboden et al., 2019. Circulation 140: 864-879), which was validated through in situ hybridization (data not shown).

Next, we identified all genes that are differentially expressed in the mouse BZ compared to the injury and remote areas using the EdgeR algorithm (data not shown). A similar analysis was performed on a previously generated TOMO-sequencing datasets of the injured zebrafish heart (data not shown. See FIG. 1B) (Wu et al., 2015. Develop Cell 36: 36-49). The differentially expressed genes in the mouse and zebrafish BZ from different timepoints (3, 7 and 14 dpi) were pooled to mitigate any temporal differences between both species. In order to compare expression of mouse and zebrafish genes, we first identified all differentially expressed BZ genes with an annotated homologue in the mouse and zebrafish genomes resulting in 11779 homologues gene-pairs. Next, we plotted for all these gene-pairs their Log Fold Change (Log FC) (BZ versus the rest of the tissue) in a scatter plot (See FIG. 1C) and selected all genes with a P-value below 0.05 and a Log FC above 0.5. This revealed 331 gene-pairs with enhanced expression in the BZ in both species and 326 gene-pairs with reduced expression in the BZ of both species. Gene ontology analysis of the 331 upregulated genes revealed that these are enriched for genes with a function in extracellular matrix, calcium binding and regulation of apoptosis, while the 326 downregulated gene-pairs have functions in mitochondria such as oxidative phosphorylation and aerobic respiration (data not shown). Together, these results indicate that both zebrafish and mouse BZ cells are responding to the injury through downregulation of their oxygen dependent metabolism and is consistent with previous studies, showing that both zebrafish and mouse BZ CMs downregulate genes with a role in oxidative phosphorylation (Honkoop et al., 2019. ELife 8: 1-27; van Duijvenboden et al., 2019. Circulation 140: 864-879; Wu et al., 2015. Develop Cell 36: 36-49).

More interesting are the observed differences between the zebrafish and mouse border zone, as they may shed light on the limited regenerative capacity of the injured mammalian heart (data not shown). We identified 366 gene-pairs that are upregulated in the mouse BZ, but not in the zebrafish BZ. These gene-pairs have functions in wound healing, extracellular matrix organization and TGFb signaling, together suggesting profound differences in the pro-fibrotic response between the zebrafish and mouse BZ (Ikeuchi et al., 2004. Cardiovascular Res 64: 526-535; Okada et al., 2005. Circulation 111: 2430-2437; Chablais & Jafwinska, 2012. Development 139: 1921-1930). Vice versa, we identified 371 gene-pairs that are upregulated in the zebrafish BZ, but not in the mouse BZ (see Table 1). For some of these genes we confirmed the BZ-enriched expression in zebrafish through in situ hybridization and the lack of BZ enrichment in the mouse BZ using quantitative PCR analysis (FIG. 2B and data not shown). The 371 gene-pairs specific for the zebrafish BZ have functions in processes such as actin filament binding and myosin complex, which may relate to a remodelling of the contractile apparatus. In addition, gene ontologies such as response to insulin stimulus are consistent with important roles for insulin signaling and metabolic reprogramming in the regulation of CM proliferation during zebrafish heart regeneration (Fukuda et al., 2020. EMBO Rep 21: e49752; Honkoop et al., 2019. ELife 8: 1-27; Y. Huang et al., 2013. PloS One 8: e67266).

In summary, we used TOMO-seq to resolve the transcriptome of the BZ of the injured adult mouse heart and compared this with the BZ of the regenerating zebrafish heart. The comparison of the BZ transcriptomes revealed processes that are shared such as reduced mitochondrial oxidative phosphorylation and processes that are species-specific such as an altered fibrotic response in mouse BZ and insulin signaling in the zebrafish BZ. Together, these results may lead to the identification of processes and pathways that promote heart regeneration.

### Hmga1a is Required for Zebrafish Heart Regeneration

From the list of zebrafish-specific BZ genes we selected hmga1a as it encodes an architectural protein that binds to and relaxes chromatin to induce and maintain cellular pluripotency and self-renewal (Battista et al., 2003. FASEB J 17: 1-27; Kishi et al., 2012. Nature Neuroscience 15: 1127-1133; Shah et al., 2012. PLoS ONE 7: e48533.; Xian et al., 2017. Nature Comm 8: 15008), suggesting an early role in regulating heart regeneration. To confirm the TOMO-seq results, we established the spatial and temporal dynamics of hmga1a/Hmga1 expression in injured zebrafish and mouse hearts by in situ hybridization. In both uninjured and 1 dpi zebrafish hearts, hmga1a expression was undetectable (data not shown). At 3 dpi hmga1a expression was weak but consistent in the BZ, which further increased at 7 dpi with robust hmga1a expression in BZ CMs. In contrast, the zebrafish paralogue hmga1b did not show any expression in BZ CMs (data not shown). In addition, Hmga1 expression was nearly undetectable in the BZ of injured adult mouse hearts (data not shown), which was confirmed by qPCR (data not shown). Next, we wondered whether Hmga1 expression correlates with the regenerative window of the regenerating neonatal mouse heart. Indeed, we did observe mosaic Hmga1 expression throughout the uninjured neonatal P1 heart (data not shown). Importantly, qPCR analysis demonstrated that Hmga1 expression levels decline rapidly in the first week after birth, coinciding with the loss of regenerative capacity of the mouse heart (FIG. 2) (Porrello et al., 2011. Science 331: 1078-1080). From these results we conclude that the temporal and spatially restricted expression of zebrafish hmga1a and mouse Hmga1, correlates well with a potential role for Hmga1 in the regeneration process.

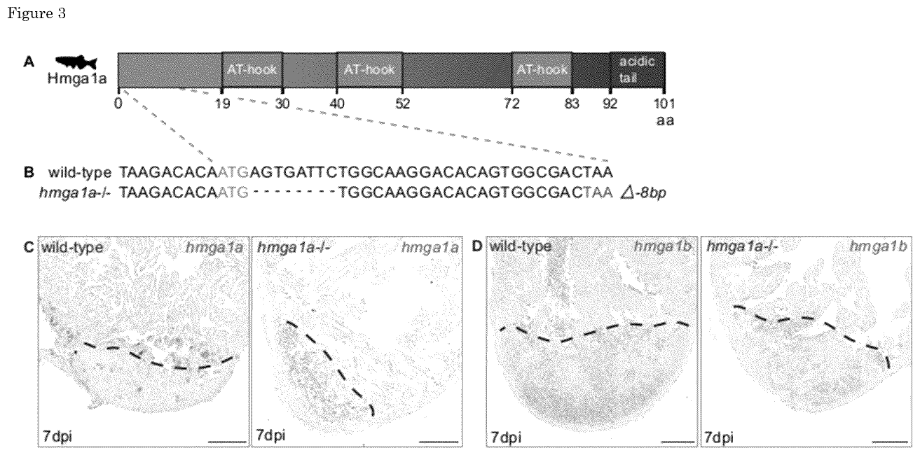

To investigate whether Hmga1 is essential for zebrafish heart regeneration, we generated a loss-of-function mutant line by targeting the start of first exon of hmga1a using a TALEN-based strategy. The resulting 8 bp deletion directly after the start-codon causes a frame-shift and introduction of a pre-mature stop codon, resulting in a truncated Hmga1a protein (7aa instead of 101aa, FIG. 3A,B). Homozygous hmga1a mutant embryos developed normally and could be grown to adulthood to study the potential role for Hmga1 during heart regeneration. Importantly, expression of hmga1a was strongly reduced in injured hearts of hmga1a−/− fish compared to their wild-type siblings, likely due to non-sense mediated mRNA decay, consistent with a loss of Hmga1a function (FIG. 3C). Upregulation of hmga1b was not observed in the hmga1a−/− hearts, suggesting hmga1b is not compensating for the loss of hmga1a (FIG. 3D).

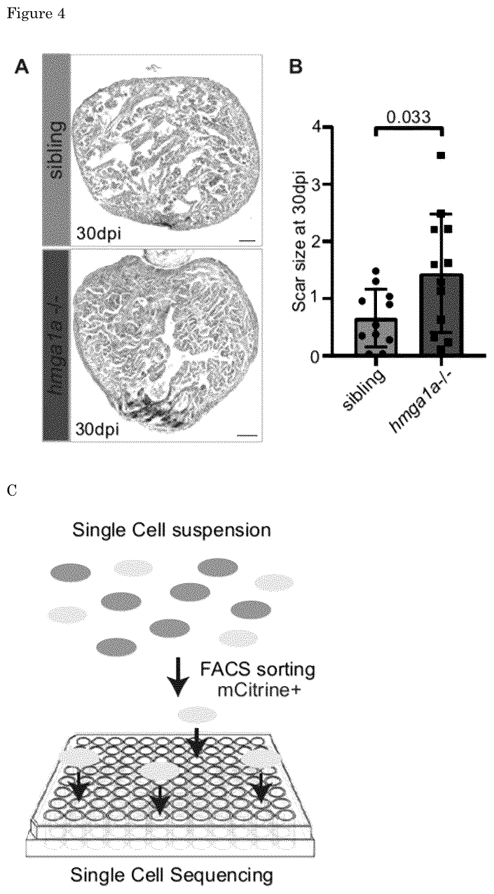

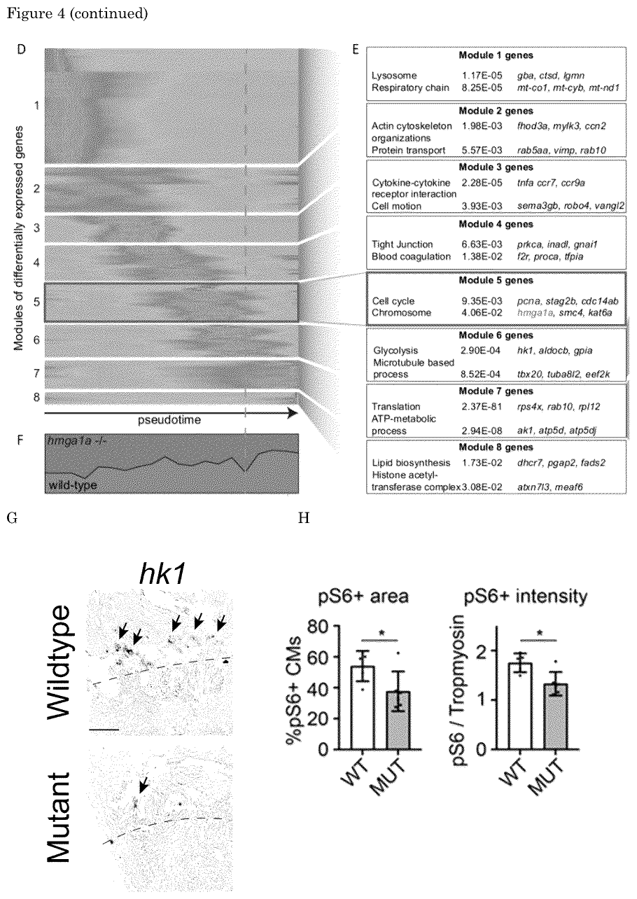

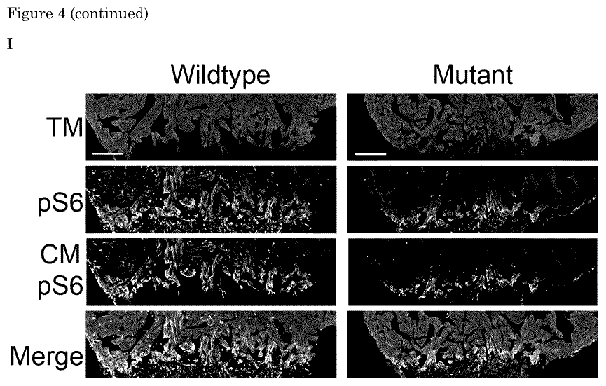

Next, we assessed whether the hmga1a−/− fish have a heart regeneration defect by visualizing fibrosis with AFOG in injured hearts 30 days post cryoinjury. Indeed, scar size at 30 dpi was significantly increased in hmga1a−/− hearts compared to their siblings, indicating impaired regeneration (FIG. 4A,B), indicating that hmga1a is required for zebrafish heart regeneration.

TABLE 1
Genes upregulated in the zebrafish border zone (BZ), but not in the mouse BZ.

| Ensemble Gene_ID | Name | Ensemble Gene_ID | Name |
|---|---|---|---|
| ENSDARG00000074254 | abcb6b | ENSDARG00000059060 | lgalsla |
| ENSDARG00000058953 | abcc4 | ENSDARG00000053535 | lmo7b |
| ENSDARG00000031795 | abcf1 | ENSDARG00000003984 | LTN1 |
| ENSDARG00000077782 | acer2 | ENSDARG00000104701 | map7d1b |
| ENSDARG00000079204 | adam11 | ENSDARG00000042551 | mboat2b |
| ENSDARG00000063079 | ago3b | ENSDARG00000088040 | si:dkeyp- |
| | | | 27c8.2 |
| ENSDARG00000056331 | ahcyl1 | ENSDARG00000008020 | med31 |
| ENSDARG00000042025 | C9H2orf49 | ENSDARG00000032318 | mfsd6a |
| ENSDARG00000009336 | aif1l | ENSDARG00000003732 | mitfa |
| ENSDARG00000005926 | ak2 | ENSDARG00000100794 | mmp13b |
| ENSDARG00000091792 | akap12a | ENSDARG00000076135 | mmrn2a |
| ENSDARG00000076544 | aldh5a1 | ENSDARG00000073711 | mmrn2b |
| ENSDARG00000077253 | alkbh6 | ENSDARG00000053453 | mpp2a |
| ENSDARG00000014969 | ankhb | ENSDARG00000010957 | mpp2b |
| ENSDARG00000002298 | ankrd22 | ENSDARG00000016573 | mroh1 |
| ENSDARG00000015589 | ankrd33bb | ENSDARG00000074602 | mrvi1 |
| ENSDARG00000020621 | ap1b1 | ENSDARG00000091001 | mycbp |
| ENSDARG00000016128 | ap3m2 | ENSDARG00000012944 | myhz2 |
| ENSDARG00000010565 | aqp4 | ENSDARG00000079686 | naa30 |
| ENSDARG00000074702 | arfgef2 | ENSDARG00000013669 | napba |
| ENSDARG00000011333 | arhgap33 | ENSDARG00000014898 | ncbp2 |
| ENSDARG00000100913 | arhgef12b | ENSDARG00000075369 | nck1b |
| ENSDARG00000006299 | arhgef7a | ENSDARG00000032849 | ndrg1a |
| ENSDARG00000070318 | arl8bb | ENSDARG00000018061 | neil1 |
| ENSDARG00000079839 | arrdc1b | ENSDARG00000040192 | nenf |
| ENSDARG00000078703 | unm_hu7910 | ENSDARG00000038687 | nfkb2 |
| ENSDARG00000021681 | asrgl1 | ENSDARG00000042627 | nhsl1b |
| ENSDARG00000036956 | asxl1 | ENSDARG00000075707 | nid2a |
| ENSDARG00000077785 | atf5b | ENSDARG00000100990 | nme3 |
| ENSDARG00000005122 | atp2a2b | ENSDARG00000043304 | nop2 |
| ENSDARG00000060978 | atp2a3 | ENSDARG00000078725 | nos1apa |
| ENSDARG00000022315 | atp6v1g1 | ENSDARG00000105071 | nos1apa |
| ENSDARG00000037009 | banf1 | ENSDARG00000012871 | npepl1 |
| ENSDARG00000090190 | bcam | ENSDARG00000102153 | nrp1a |
| ENSDARG00000016231 | bcap29 | ENSDARG00000043209 | nsun5 |
| ENSDARG00000070864 | bcl6aa | ENSDARG00000094647 | BX465844.1 |
| ENSDARG00000040396 | bcl7bb | ENSDARG00000059646 | nt5dc2 |
| ENSDARG00000059388 | bdh1 | ENSDARG00000104837 | nudc |
| ENSDARG00000032369 | btbd6b | ENSDARG00000052336 | ociad2 |
| ENSDARG00000076401 | cacng3b | ENSDARG00000004634 | osbp |
| ENSDARG00000014731 | cacybp | ENSDARG00000034189 | oxsr1a |
| ENSDARG00000057013 | cadm3 | ENSDARG00000076966 | pagr1 |
| ENSDARG00000040291 | cadm4 | ENSDARG00000010583 | pard3ab |
| ENSDARG00000036344 | calb2b | ENSDARG00000044625 | pcf11 |
| ENSDARG00000013804 | capns1b | ENSDARG00000087386 | None |
| ENSDARG00000018698 | carm1 | ENSDARG00000073985 | pctp |
| ENSDARG00000036164 | cars1 | ENSDARG00000045305 | pde7a |
| ENSDARG00000103747 | cav1 | ENSDARG00000055477 | pelo |
| ENSDARG00000074337 | cbfa2t2 | ENSDARG00000025391 | pfdn2 |
| ENSDARG00000038025 | cbx7a | ENSDARG00000062363 | phex |
| ENSDARG00000062307 | ccdc61 | ENSDARG00000038737 | phf20b |
| ENSDARG00000077938 | cd248b | ENSDARG00000042874 | phlda2 |
| ENSDARG00000037473 | cd79a | ENSDARG00000079378 | phldb1b |
| ENSDARG00000058943 | cdcp1a | ENSDARG00000062445 | pias1b |
| ENSDARG00000056683 | cdk5 | ENSDARG00000076870 | piezo1 |
| ENSDARG00000035577 | cds2 | ENSDARG00000075530 | pigu |
| ENSDARG00000069185 | celsr1a | ENSDARG00000004527 | pin4 |
| ENSDARG00000062152 | chaf1a | ENSDARG00000063313 | plbd1 |
| ENSDARG00000075211 | chd7 | ENSDARG00000079572 | plcd3b |
| ENSDARG00000100304 | chdh | ENSDARG00000062590 | pleca |
| ENSDARG00000041078 | chka | ENSDARG00000102435 | plekhf1 |
| ENSDARG00000091321 | CABZ01088229.1 | ENSDARG00000070174 | PLEKHH3 |
| ENSDARG00000004037 | cog2 | ENSDARG00000099954 | plekhm1 |
| ENSDARG00000029660 | commd5 | ENSDARG00000007172 | plxna3 |
| ENSDARG00000011769 | cpm | ENSDARG00000077469 | polr1b |
| ENSDARG00000031968 | cpped1 | ENSDARG00000036625 | polr2f |
| ENSDARG00000020217 | cpsf4 | ENSDARG00000040443 | polr2i |
| ENSDARG00000074758 | csde1 | ENSDARG00000075616 | polr2k |
| ENSDARG00000043658 | cxadr | ENSDARG00000031317 | ppdpfb |
| ENSDARG00000076742 | cyth1a | ENSDARG00000015422 | ppil1 |
| ENSDARG00000077926 | si:dkey- | ENSDARG00000074690 | ppm1j |
| | 48p11.3 | | |
| ENSDARG00000017985 | C1H4orf33 | ENSDARG00000102009 | ppp2r2d |
| ENSDARG00000068602 | dalrd3 | ENSDARG00000045540 | ppp6r2a |
| ENSDARG00000041363 | dctn3 | ENSDARG00000003818 | prkag1 |
| ENSDARG00000032117 | ddx1 | ENSDARG00000034173 | prkcq |
| ENSDARG00000030789 | ddx18 | ENSDARG00000033126 | prkrip1 |
| ENSDARG00000104793 | dgke | ENSDARG00000042489 | thap12a |
| ENSDARG00000014956 | diablob | ENSDARG00000015239 | prpf19 |
| ENSDARG00000016484 | dkc1 | ENSDARG00000033971 | prrx1a |
| ENSDARG00000045219 | dkk1b | ENSDARG00000016733 | psat1 |
| ENSDARG00000052072 | dnajb9a | ENSDARG00000013938 | psmb3 |
| ENSDARG00000016886 | dnajb9b | ENSDARG00000054696 | psmb4 |
| ENSDARG00000097601 | CT868708.1 | ENSDARG00000023279 | psmd4b |
| ENSDARG00000020676 | dpp3 | ENSDARG00000040620 | psmg1 |
| ENSDARG00000037652 | C1H7orf26 | ENSDARG00000090191 | None |
| ENSDARG00000057323 | e2f8 | ENSDARG00000035986 | ptpn2b |
| ENSDARG00000010432 | eaf2 | ENSDARG00000059362 | cavin1b |
| ENSDARG00000061377 | efcab1 | ENSDARG00000045562 | pus1 |
| ENSDARG00000074323 | efcab2 | ENSDARG00000056347 | rab3aa |
| ENSDARG00000074050 | efnb2b | ENSDARG00000028389 | rab44 |
| ENSDARG00000079895 | ehbp1l1b | ENSDARG00000043593 | rapgef1a |
| ENSDARG00000029445 | eif1b | ENSDARG00000014746 | rbfox1 |
| ENSDARG00000016889 | eif3g | ENSDARG00000012723 | rbm14a |
| ENSDARG00000053370 | eif3jb | ENSDARG00000038030 | rbx1 |
| ENSDARG00000031819 | eif4ebp2 | ENSDARG00000008278 | rcor2 |
| ENSDARG00000056186 | eif5a2 | ENSDARG00000035810 | rgcc |
| ENSDARG00000035282 | emc6 | ENSDARG00000079816 | rhbdd3 |
| ENSDARG00000008808 | eml2 | ENSDARG00000016830 | rimkla |
| ENSDARG00000058865 | endog | ENSDARG00000036282 | rnaset2 |
| ENSDARG00000013750 | eno1b | ENSDARG00000060035 | rnf32 |
| ENSDARG00000038422 | entpd4 | ENSDARG00000080410 | snrnau11 |
| ENSDARG00000008377 | epn2 | ENSDARG00000083417 | CR385068.4 |
| ENSDARG00000013997 | ern1 | ENSDARG00000009387 | robo4 |
| ENSDARG00000103980 | ets2 | ENSDARG00000002613 | rpa3 |
| ENSDARG00000063211 | exoc8 | ENSDARG00000041991 | rrp9 |
| ENSDARG00000025091 | ezrb | ENSDARG00000078125 | rusc1 |
| ENSDARG00000090063 | fa2h | ENSDARG00000060319 | scn4bb |
| ENSDARG00000088073 | fam110b | ENSDARG00000022996 | setx |
| ENSDARG00000043339 | abraxas1 | ENSDARG00000025522 | sgk1 |
| ENSDARG00000105040 | dipk1c | ENSDARG00000062315 | sik2b |
| ENSDARG00000034497 | fbxl14a | ENSDARG00000073756 | slc12a7a |
| ENSDARG00000006924 | fbxo38 | ENSDARG00000032010 | slc15a2 |
| ENSDARG00000004782 | fgfr3 | ENSDARG00000006760 | slc24a3 |
| ENSDARG00000057004 | si:ch211- | ENSDARG00000057287 | slc25a16 |
| | 287a12.9 | | |
| ENSDARG00000076120 | foxp4 | ENSDARG00000005463 | slc30a1a |
| ENSDARG00000101518 | CR854829.1 | ENSDARG00000038106 | slc37a4a |
| ENSDARG00000102626 | frem2b | ENSDARG00000009901 | slc38a5a |
| ENSDARG00000079730 | fuz | ENSDARG00000057949 | slc43a3b |
| ENSDARG00000015537 | gad2 | ENSDARG00000010816 | slc7a14a |
| ENSDARG00000059070 | gars1 | ENSDARG00000036096 | smad3a |
| ENSDARG00000027612 | gatad1 | ENSDARG00000020730 | smpd4 |
| ENSDARG00000056122 | gdi1 | ENSDARG00000016086 | smurf1 |
| ENSDARG00000038537 | gga1 | ENSDARG00000077536 | snrnp200 |
| ENSDARG00000028106 | glrx | ENSDARG00000008395 | snupn |
| ENSDARG00000098785 | glrx3 | ENSDARG00000037476 | sorbs3 |
| ENSDARG00000025826 | gna12a | ENSDARG00000098834 | sox4b |
| ENSDARG00000037921 | gng13b | ENSDARG00000076763 | sp2 |
| ENSDARG00000039830 | gng5 | ENSDARG00000004017 | spag1a |
| ENSDARG00000074401 | gnpat | ENSDARG00000024933 | spast |
| ENSDARG00000091931 | gpatch4 | ENSDARG00000052624 | spryd7a |
| ENSDARG00000090585 | gpc1b | ENSDARG00000053918 | srfa |
| ENSDARG00000016011 | gpcpd1 | ENSDARG00000031560 | srpk1b |
| ENSDARG00000005414 | grapa | ENSDARG00000088440 | ssh2a |
| ENSDARG00000037496 | gria4a | ENSDARG00000036584 | st8sia5 |
| ENSDARG00000062020 | gse1 | ENSDARG00000098883 | stac3 |
| ENSDARG00000015681 | gsk3ab | ENSDARG00000043281 | stap2b |
| ENSDARG00000030340 | guk1a | ENSDARG00000007603 | stxbp2 |
| ENSDARG00000036076 | heatr3 | ENSDARG00000058220 | tada3l |
| ENSDARG00000101482 | hk2 | ENSDARG00000013250 | tars1 |
| ENSDARG00000055991 | hmbsb | ENSDARG00000061986 | tbc1d2b |
| ENSDARG00000028335 | hmga1a | ENSDARG00000022918 | tbcc |
| ENSDARG00000099312 | jpt1b | ENSDARG00000052344 | tbl3 |
| ENSDARG00000018944 | hoga1 | ENSDARG00000078186 | tex2 |
| ENSDARG00000042946 | hook1 | ENSDARG00000034895 | tgfb1b |
| ENSDARG00000018397 | hpca | ENSDARG00000061508 | tgfbrap1 |
| ENSDARG00000015749 | hps3 | ENSDARG00000042659 | thyn1 |
| ENSDARG00000071501 | hs6st1b | ENSDARG00000056259 | tmem131 |
| ENSDARG00000071377 | hsd11b1la | ENSDARG00000079272 | None |
| ENSDARG00000068992 | hspa8 | ENSDARG00000079858 | tmem163a |
| ENSDARG00000056649 | htatsf1 | ENSDARG00000070455 | tmem245 |
| ENSDARG00000032831 | htra1a | ENSDARG00000031956 | tmem63a |
| ENSDARG00000014907 | htra1b | ENSDARG00000060498 | tnfrsf9a |
| ENSDARG00000059725 | hypk | ENSDARG00000057241 | tnfsf10 |
| ENSDARG00000040764 | id1 | ENSDARG00000087402 | tpm1 |
| ENSDARG00000096939 | CU207354.1 | ENSDARG00000010445 | trabd |
| ENSDARG00000054906 | ier5l | ENSDARG00000060729 | trim8b |
| ENSDARG00000038879 | ift80 | ENSDARG00000069278 | trmt5 |
| ENSDARG00000033307 | igf2b | ENSDARG00000008480 | trmt61a |
| ENSDARG00000022176 | IGLON5 | ENSDARG00000089986 | None |
| ENSDARG00000068711 | crfb4 | ENSDARG00000007918 | ttc27 |
| ENSDARG00000089131 | il17rel | ENSDARG00000044405 | ttc4 |
| ENSDARG00000104693 | il6st | ENSDARG00000060374 | ttll11 |
| ENSDARG00000068175 | ing5b | ENSDARG00000000563 | ttn.1 |
| ENSDARG00000101032 | iqcb1 | ENSDARG00000052170 | uap1 |
| ENSDARG00000069844 | acod1 | ENSDARG00000040286 | ubl7b |
| ENSDARG00000078717 | itga8 | ENSDARG00000022340 | ufc1 |
| ENSDARG00000044318 | itgb7 | ENSDARG00000020228 | usf2 |
| ENSDARG00000061741 | itpr3 | ENSDARG00000103422 | usp10 |
| ENSDARG00000010252 | jak3 | ENSDARG00000032327 | usp36 |
| ENSDARG00000021827 | katna1 | ENSDARG00000044575 | vars1 |
| ENSDARG00000069271 | kbtbd4 | ENSDARG00000040466 | vil1 |
| ENSDARG00000055855 | kcnc3a | ENSDARG00000059079 | vps25 |
| ENSDARG00000056101 | kcnd3 | ENSDARG00000039270 | vti1b |
| ENSDARG00000045067 | kcnk1a | ENSDARG00000095879 | wdr46 |
| ENSDARG00000054978 | kifc3 | ENSDARG00000007990 | wt1b |
| ENSDARG00000061368 | klf13 | ENSDARG00000015966 | yaf2 |
| ENSDARG00000100206 | klhdc4 | ENSDARG00000016447 | ythdf1 |
| ENSDARG00000019125 | klhl40b | ENSDARG00000039899 | zbtb7a |
| ENSDARG00000038066 | kpna2 | ENSDARG00000016368 | zbtb8os |
| ENSDARG00000052641 | kpna3 | ENSDARG00000075165 | zc3h6 |
| ENSDARG00000076464 | lamtor1 | ENSDARG00000018936 | zcchc17 |
| ENSDARG00000013741 | lancl1 | ENSDARG00000009178 | zfand1 |
| ENSDARG00000101722 | larp1 | ENSDARG00000060113 | znf395a |
| ENSDARG00000034896 | ldb2b | ENSDARG00000052894 | znf532 |
| ENSDARG00000015824 | lemd3 | ENSDARG00000027403 | zgpat |
| | | ENSDARG00000030805 | znhit2 |

### Himga1a is Required for Cardiomyocyte Proliferation

To gain insight into the function of hmga1a during heart regeneration we aimed to identify which processes occurring in BZ CMs dependent on hmga1a. To accomplish this, we crossed the hmga1a−/− mutants with the previously published nppa:mCitrine reporter line which marks BZ CMs (Honkoop et al., 2019. ELife 8: 1-27). This allowed us to sort out BZ CMs (mCitrine high) of 7 dpi hmga1a−/− and sibling hearts using FACsorting. Next, we submitted the isolated cells to single-cell RNA-sequencing using the SORT-seq (SOrting and Robot-assisted Transcriptome SEQuencing) platform (FIG. 4C) (Muraro et al., 2016. Cell Systems 3: 385-394). We analysed 1311 cells with over 600 reads per cell using the Race-ID3 algorithm resulted in the identification of 6 cell clusters grouped based on their transcriptomic features. All analysed cells contain reads of the cardiomyocyte marker myl7, validating that we specifically sorted out CMs. In addition, all clusters with the exception of cluster 5 show expression of BZ markers nppa and desma, indicating that these cells represent BZ CMs. Cluster 5 cells instead express a high level of mitochondrial mRNAs like NC_002333.17 suggesting a more remote origin (data not shown).

To investigate where the hmga1a−/− cells are represented in the t-SNE map, we plotted the genotype specific contribution. Quantification of the genotype contribution per cluster (FIG. 4D) indicates that while most clusters are slightly enriched for hmga1a−/− cells, clusters 1 and 6 are almost exclusively formed by wild-type CMs. We validated this by in situ hybridization for the cluster 6-enriched genes fth1a, uba52 and gamt (data not shown) and indeed observed reduced expressed of these genes in the BZ CMs of hmga1a−/− hearts. To address transcriptional differences between cell clusters we performed gene ontology analysis on cluster-enriched genes. Importantly, cluster 1 and 6 specific genes are enriched in genes with a role in cell cycle regulation, suggesting that CM proliferation is affected in hmga1a−/− hearts.

To gain insight into the processes regulated by Hmga1a we performed unsupervised pseudo-time ordering of the single cells. Next, gene expression profiles were unbiasedly grouped together in self organizing modules (SOMs) of similarly expressed genes, resulting in 8 transcriptionally distinct modules (data not shown; see FIG. 4E). Module 1 represents genes with high expression at the start of the pseudo-time line with a role in the respiratory chain, indicating that this represents the most differentiated CM state. Importantly, module 5 contains hmga1a as well as genes with a role in cell cycle control (e.g. pcna and cdc14), consistent with a close correlation between Hmga1a and CM proliferation. Next, we wondered how hmga1a−/− cells are distributed across the pseudo time line. To this end, we plotted the relative genotype contribution across 100 bins constituting the pseudo time. Interestingly, we observe a trend in which there is a gradual loss of hmga1a−/− cell contribution as pseudo time progresses. Strikingly, after module 5, the number of hmga1a−/− cells contributing to the pseudo-timeline drops from constituting ˜50% (during module 5) to ˜28% (modules 6-8). Directly preceding the drop in mutant cell contribution, a peak in mutant cell contribution is observed at the edge of module 5 (data not shown). These data suggest that hmga1a−/− BZ CMs fail to progress in pseudo time, but instead are arrested at earlier stages. Indeed, in situ hybridization for hk1 (module 6 marker) and ak1 (module 7 marker) revealed reduced expression in hmga1a−/− hearts at 7 dpi (FIG. 4G; data not shown). The co-expression of hmga1a with cell cycle regulators and reduced expression of hk1, encoding the rate-limiting glycolysis enzyme hexokinase, in hmga1a−/− hearts further suggested that Hmga1a is specifically required for cell cycle induction and/or progression.

Modules 7 and 8 consisted mainly of genes involved in translation and re-employment of an oxidative metabolism. Increased rates of translation and oxidative phosphorylation are correlated to cell cycle progression towards mitosis. Impaired induction of these various genes in hmga1a mutants may well explain the observed reduction in CM proliferation. To validate whether translation is affected in injured hmga1a mutant hearts we first assessed levels of phosphorylated-S6 (pS6, Ser253/263) in border zone CMs as a readout of translation (FIG. 4I). The ribosomal S6 protein is part of the 40S ribosomal subunit and phosphorylation at its Ser235/Ser236 residues promotes translation initiation rates. An increase in pS6 was observed in the border zone yet not restricted to myocardial cells. Hence, we performed masking using a Tropomyosin counterstain to subset only the myocardial contribution. We observed that both the area and intensity of myocardial pS6 was significantly reduced in the mutant border zone (FIG. 4H), implying that translation rates are affected in border zone CMs in hmga1a mutant hearts. Together, these results indicate that Hmga1a is important to activate cell cycle reentry, a metabolic shift towards glycolysis and protein translation.

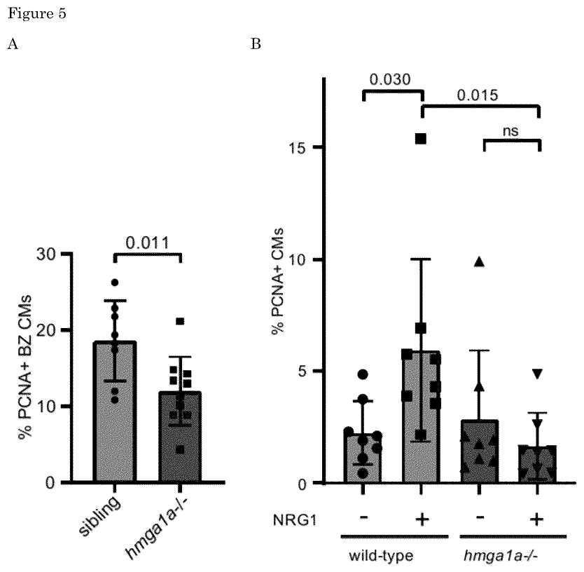

To validate this, we assessed BZ CM proliferation through immunohistochemistry with PCNA and indeed observed a significant decrease in PCNA+ CMs in hmga1a−/− hearts compared to their siblings (FIG. 5A). Taken together, these data indicate that hmga1a promotes CM proliferation in the BZ, while it is dispensable for the early response of the BZ CMs to the injury.

### Hmga1a Acts Downstream of Nrg1 Signaling

The Nrg1 growth factor, which is produced and secreted by epicardial derived cells in response to heart injury, induces CM proliferation (Gemberling et al., 2015. ELife 4: 1-17). As we observed that BZ CM proliferation requires Hmga1a, we addressed whether Nrg1 induces hmga1a expression and whether Hmga1a is also required for Nrg1-induced CM proliferation. Therefore, we injected fish intraperitoneal with human recombinant NRG1, once a day for three consecutive days. Similar as reported for a genetic nrg1 overexpression model (Gemberling et al., 2015. ELife 4: 1-17), increased cardiomyocyte proliferation was observed upon NRG1-injection, which was most pronounced in the outermost myocardial layers of the heart (data not shown). Furthermore, ectopic NRG1 induced hmga1a expression throughout the entire heart, including the outermost myocardial layers (data not shown). To investigate whether NRG1-induced CM proliferation is dependent on hmga1a, we injected NRG1 in hmga1a−/− fish. Importantly, while NRG1 induced CM proliferation in wild-type siblings, ectopic NRG1 failed to induce CM proliferation in hmga1a−/− hearts (FIG. 5B). Together, these results indicate that hmga1 acts downstream of Nrg1 signaling to induce CM proliferation. Hmga1a changes chromatin accessibility and induces an injury-related gene program. Hmga1 is an architectural chromatin protein that preferentially binds to AT-rich domains and in vitro experiments have demonstrated that Hmga1 competes for DNA binding with Histone H1 (Catez et al., 2004. Mol Cell Biol 24: 4321-4328). Upon chromatin binding, Hmga1 facilitates binding of chromatin remodelling complexes and transcription factors to induce gene transcription (Catez et al., 2004. Mol Cell Biol 24: 4321-4328; Ozturk et al., 2014. Front Cell Develop Biol 2: 5; Reeves, 2001. Gene 277: 63-81). To reveal whether hmga1a overexpression in zebrafish CMs leads to changes in chromatin accessibility and gene expression we generated a Tg(ubi:Loxp-stop-Loxp-hmga1a-eGFP) line, which, when crossed with Tg(myl7:CreERT2) combined with tamoxifen treatment results in CM-specific overexpression of Hmga1a-eGFP. We isolated nuclei from hmga1a overexpression and Cre-only control hearts 14 days post recombination and FACsorted CM nuclei using the CM nuclear reporter line Tg(myl7:DsRed-nls). Next, CM nuclei were subjected to an assay for transposase-accessible chromatin using sequencing (ATAC-seq) (Buenrostro et al., 2013. Nat Methods 10: 1213-1218), resulting in 311,968,790 sequencing reads that were mapped to the zebrafish genome (GRCz10). Genomic regions were mapped using Bowtie2 and accessible regions were identified using MACS2 peak calling. In total, we identified 35,014 accessible regions in the hmga1a overexpression dataset and 37,718 accessible regions in the control dataset (q-value>0.05, calculated with Benjamini-Hochberg procedure; Benjamini and Hochberg, 1995. J R Stat Soc B 57: 289-300). Most accessible regions are in inter- and intragenic regions, while a smaller fraction was found in promotor regions (data not shown). A large proportion of these accessible promotor regions (n=1,308) are shared between the control and hmga1a overexpression datasets (data not shown). We identified 12,906 emerging and 15,855 disappearing accessible chromatin regions upon hmga1a overexpression, while 22,430 chromatin regions remained stably accessible. As these results support a role for Hmga1a in changing chromatin accessibility we next wanted to know whether these regions are correlated with specific transcription factors that may bind to these regions. Therefore, we identified enriched transcription factor binding motifs within the emerging and disappearing peaks, using the web based MEME suite tool Analysis of Motif Enrichment (AME v5.3.0) (Bailey et al., 2009. Nucleic Acids Res 43: W39-W49; McLeay & Bailey, 2010. BMC Bioinformatics. 11: 165). Strikingly, both emerging and disappearing peaks showed strong enrichment in binding motifs for the transcription factors NF-YA, FOXK1 and FOXK2, indicating that Hmga1a overexpression modulates their respective binding sites (data not shown).

To investigate the correlation between promotor accessibility and transcription we also performed RNA-seq on hmga1a overexpressing- and control-hearts and integrated these data with the ATAC-seq data. We found that promoter accessibility and transcription are well correlated for both control and hmga1a overexpression datasets, as a significantly higher RNA-expression was observed for genes with accessible promotor regions (data not shown). In hmga1a overexpression hearts 1,647 genes are upregulated (p.value<0.05, Log FC>0.5), including genes with a role in DNA replication, corroborating a role for Hmga1a in cell proliferation. Surprisingly, the natriuretic peptide genes are also induced by hmga1a overexpression. This is striking as it indicates induction of an injury responsive program, while the hearts used for the analysis had not been injured. This is consistent with the downregulation of many genes with functions in mitochondrial oxidative phosphorylation. As these adaptations are highly reminiscent of the processes occurring in the zebrafish BZ after injury, we compared hmga1a overexpression transcriptomes with the BZ transcriptomes. Strikingly, we observed a significant overlap for hmga1a-induced genes and genes upregulated in the injury BZ (11.5% overlap, p=6.6e-5) as well as for hmga1a-repressed genes and genes downregulated in the injury BZ (18.2% overlap, p=4.0e-11) (Bailey et al., 2009. Nucleic Acids Res 43: W39-W49; McLeay & Bailey, 2010. BMC Bioinformatics. 11: 165). In summary, these results demonstrate that hmga1a expression in CMs leads to profound changes in chromatin accessibility and subsequently the regulation of a gene-expression program that correlates with the injury-induced gene expression program found in the zebrafish BZ.

### Hmga1a Overexpression is Sufficient to Drive Proliferation in Zebrafish Cardiomyocytes

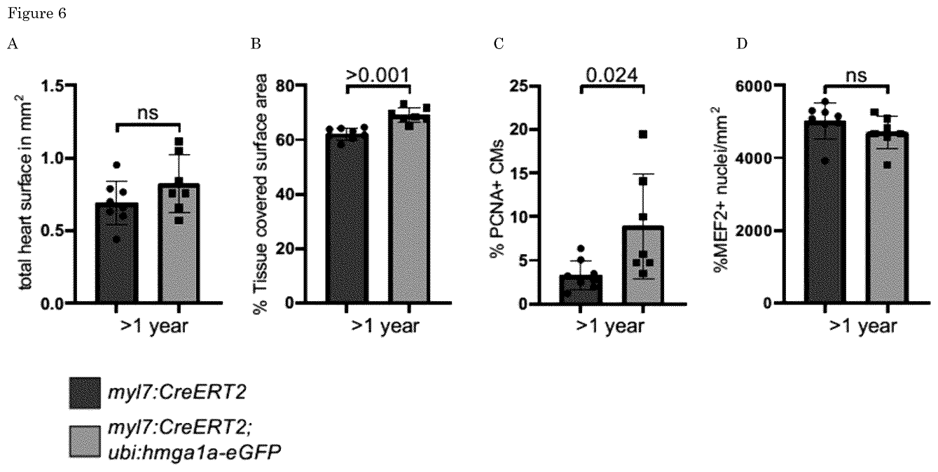

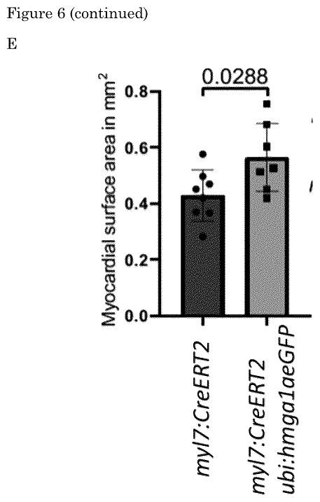

As hmga1a overexpression induces an injury-related gene expression program we wanted to investigate the effects of hmga1a overexpression on heart morphology. First, we addressed whether overexpression of hmga1a is sufficient to drive proliferation in zebrafish CMs. To analyse the effect of hmga1a overexpression we treated 4- to 6-month old Tg(ubi:Loxp-stop-Loxp-hmga1a-eGFP, myl7:CreERT2) and Tg(myl7:CreERT2) control fish with tamoxifen and extracted hearts 14 days post induction, followed by quantification of CM proliferation by PCNA expression. This revealed a significant increase in PCNA+ CMs throughout the heart from ˜7% in controls to ˜18% in hmga1a overexpressing hearts (data not shown). Next, we determined long-term effects of hmga1a overexpression and analysed hearts 1 year post induction. We observed a significant increase in myocardial surface area (data not shown), which coincided with a slight, but non-significant, increase in total surface area (including cardiac lumen) of the heart (FIG. 6A). This increase in myocardial surface area is mainly due to an expansion of the trabecular region at the expense of the cardiac lumen (FIG. 6B). Furthermore, the increase trabecular tissue is likely due to the modest but significant increase in CM proliferation (FIG. 6C), but not due an increase in CM size (FIG. 6D).

Activation of genomic regions related to heart development in Hmga1a overexpression OE cardiomycoytes was paralleled by altered histone modifications. Data for H3k4me3, indicating tri-methylation at the 4^{th} lysine residue of the histone H3 protein (activating histone mark) and H3k27me3, indicating tri-methylation of lysine 27 on histone H3 protein (repressive histone mark) were obtained by performing bulk (100 cells) sort assisted single-cell chromatin immunocleavage (sortChic; Zeller et al., 2021. bioRxiv 10.1101/2021.04.26.440606) on control cardiomyocytes (myl7:LifeAct-tdTomato+) and Hmga1a OE cardiomyocytes (myl7:LifeAct-tdTomato+/myl7:Hmga1a-EGFP+) respectively. Reads were mapped against the zebrafish reference genome (danRer10) and visualized using Galaxy. Unbiased MACS2 peak calling was subsequently performed on H3k4me3 tracks to identify regions enriched for this histone mark. Peaks on promotor regions of the embryonic cardiac transcription factors tbx5 and nkx2.5 were only called in Hmga1a OE cardiomyocytes indicating that embryonic cardiac genes are re-expressed in Hmga1a OE cardiomyocytes. Together, these results show that hmga1a overexpression induces CM proliferation, which has profound effects on heart morphology.

Hmga1 Induces Mammalian Cardiomyocyte Cell Cycle Re-Entry and Functional Recovery after Myocardial Infarction

Our previous results demonstrate that in zebrafish Hmga1a is required for efficient heart regeneration and that Hmga1a overexpression is sufficient to induced CM proliferation. Unlike in zebrafish, murine Hmga1 expression is not induced by a cardiac injury, which may be explained by the absence of an Nrg1-induced activation of ErbB2 signaling (D'Uva et al., 2015. Nat Cell Biol 17: 627-638). Therefore, we first wanted to investigate whether activation of ErbB2 signaling in murine cardiomyocytes induces Hmga1 expression. Indeed, in mouse hearts expressing the constitutively active (ca) ERBB2 receptor, we observed enhanced nuclear Hmga1 protein accumulation (data not shown).

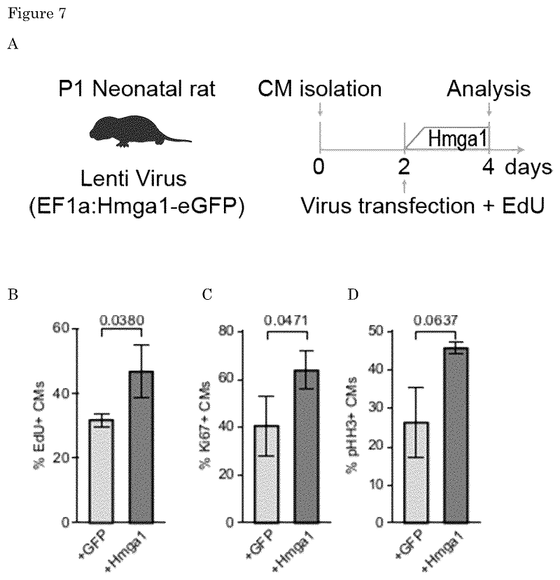

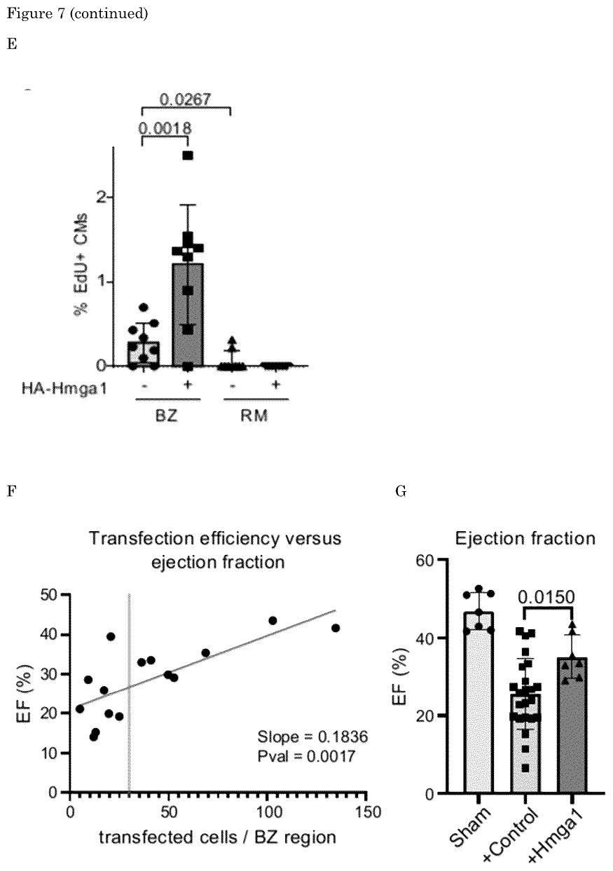

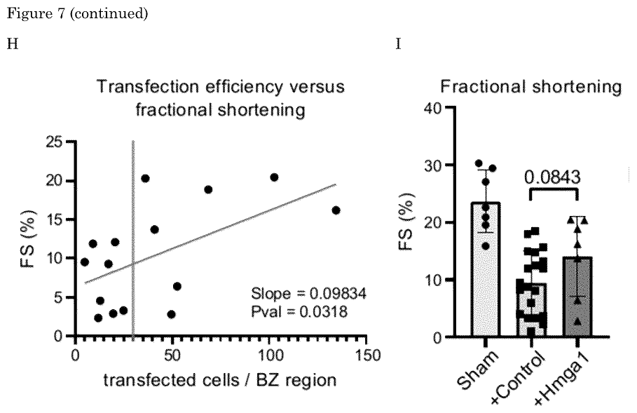

To address whether Hmga1 is able to stimulate proliferation in mammalian CMs, we induced ectopic Hmga1 expression in primary isolated neonatal rat cardiomyocytes (NRCMs) (FIG. 7A). CMs were isolated from P1 neonatal rats and incubated for 2 days before virus treatment. Hmga1-eGFP overexpressing virus (EF1a:Hmga1-eGFP) or GFP only control virus (EF1a:eGFP) was administered in the medium, as well as EdU to track newly synthesized DNA. After another 2 days of incubation, cells were fixed and analyzed for proliferation markers.

Interestingly, a significant increase in EdU and Ki67 was observed in the Hmga1-eGFP transfected CMs compared to the eGFP only transfected CMs (FIG. 7B, C). In addition, a positive trend was found for an increase in mitosis marker pHH3 in the Hmga1-eGFP transfected cells (p.val.=0.064) (FIG. 7D). Taken together, these results indicate that Hmga1 overexpression in mammalian CMs can increase their proliferative capacity.

Next we wanted to address whether ectopic Hmga1 expression in adult mouse hearts induces CM cell cycle re-entry in vivo. To induce Hmga1 expression, we employed adeno-associated virus 9 (AAV9), which preferentially targets CMs (Bish et al., 2008. Human Gene Therapy 19: 1359-1368), carrying a CMV:HA-Hmga1 cassette. Upon MI, hearts were injected twice with 15 μl virus (1×10\hat{ }¹¹ virus particles/mouse) in regions bordering the area at risk of ischemic injury. In addition, to assess CM cell cycle activity mice were injected with EdU bidaily for two weeks post MI. At 14 days post MI and virus injection, hearts were isolated, sectioned and stained for EdU (indicating newly synthesized DNA), PCM-1 (marking CM nuclei) and the HA-tag (marking cells with HA-Hmga1 overexpression) (data not shown). Importantly, while EdU incorporation in CMs located in the remote area was not affected by HA-Hmga1 expression, EdU incorporation in CMs located at the BZ was increased 5-fold in CMs expressing HA-Hmga1 (from 0.2% to 1.2%) (FIG. 7E).

Next, we wondered whether overexpression of HA-Hmga1 can lead to a functional improvements post MI. Therefore, virus treated mice were subjected to echocardiography at 42 dpi, after which the hearts were isolated and sectioned for histological analysis. Immunohistochemistry against HA-tag was performed to assess the transfection efficiency in the border zone. As the transfection efficiency showed high variability between hearts, correlation analysis between transfection efficiency and scar sizes as well as heart functionality was performed. In addition, inefficiently transfected hearts (average of <30 transfected BZ cells) were excluded when comparing between AAV9(empty) and AAV9(CMV:HA-Hmga1) treated hearts. Significant correlations were found between transfection efficiency and ejection fraction as well as fractional shortening, where hearts with a higher number of transfected BZ cells showed improved functionality (FIG. 7F,H). Direct comparison between AAV9(empty) and effectively transfected AAV9(CMV:HA-Hmga1) treated hearts indicates a significant increase in ejection fraction (p.val=0.015) (FIG. 7G), as well as a positive trend in fractional shortening (p.val.=0.084) (FIG. 71). Together, these results indicate that ectopic Hmga1 expression in injured mammalian hearts promotes cardiomyocyte cell cycle re-entry and suggests that this stimulates functional recovery post myocardial infarction.

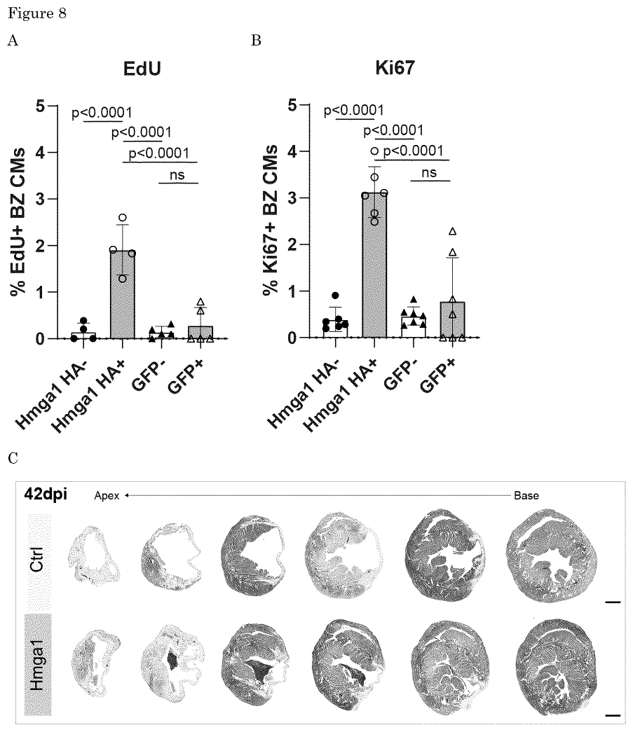

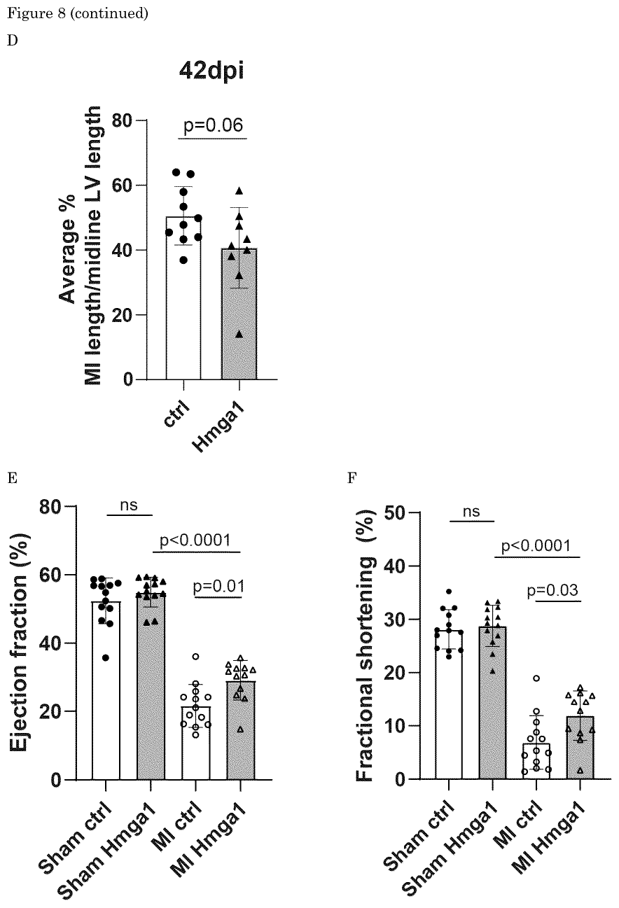

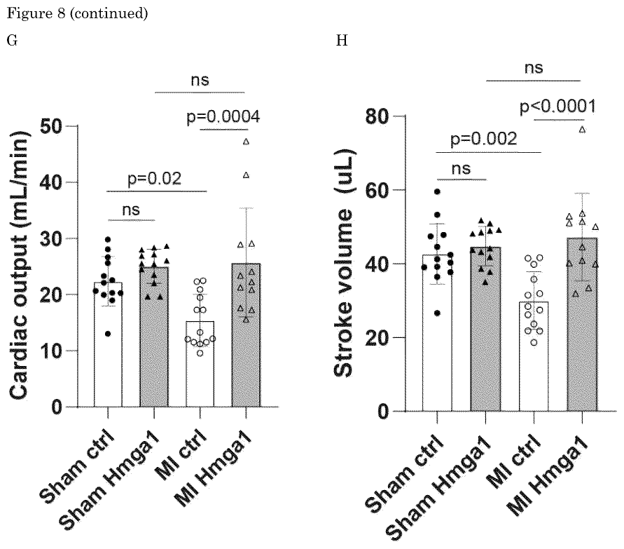

To address whether a higher transfection efficiency would result in a better functional recovery, a 10 times higher dose of AAV9(CMV:HA-Hmga1) virus (1×10\hat{ }12 virus particles/mouse) was injected into the BZ of mouse hearts after inducing MI. In addition, AAV9(CMV:HA-Hmga1) virus was injected in hearts of sham operated mice and a AAV9(CMV:GFP) virus was used as a control virus. Cardiomyocyte proliferation was further analyzed at 14 days post MI and intracardiac delivery of AAV9(CMV:HA-Hmga1) or AAV9(CMV:GFP) control virus. Antibody staining against PCM-1 (marking CM nuclei), HA (marking transfected cells expressing HA-Hmga1) and EdU (indicating newly synthesized DNA) showed EdU+(FIG. 8A) or Ki67+(FIG. 8B) cells within the border zone (BZ) of hearts transduced with HA-Hmga1 or GFP. 3 heart sections were quantified per heart. These data clearly show that ectopic Hmga1 expression in injured mammalian hearts promotes cardiomyocyte cell cycle re-entry for at least 14 days.

To address whether the increase in CM proliferation leads to improved function and reduced scar size the mice injected with AAV9(CMV:HA-Hmga1) virus or AAV9(CMV:GFP) control virus were subjected to echocardiography after which the hearts were extracted for histological analysis. Hearts were sectioned from apex to base stained using Masson's trichrome to visualize the fibrotic scar. Scar size was quantified using two independent methods. In the first method two lines were drawn from the boundaries of the scar with the myocardium to the middle of the heart lumen and their angle was measured in serial sections (data not shown). In the second method the length of the scar was measured and divided by the length of the unaffected wall of the left ventricle (FIG. 8D). Both methods showed that scar size in MI hearts injected with the AAV9(CMV:HA-Hmga1) virus was smaller in comparison with the scars formed in MI hearts injected with the AAV9(CMV:GFP) control virus. The echocardiography data were used to measure functional recovery. At 42 days post MI, significant improvements in cardiac ejection fraction (FIG. 8E), fractional shortening (FIG. 8F), cardiac output (FIG. 8G) and stroke volume (FIG. 8H) were observed in MI hearts injected with AAV9(CMV:HA-Hmga1) virus compared to MI hearts injected with AAV9(CMV:GFP) control virus. Together these results demonstrate that ectopic Hmga1 expression in injured mammalian hearts promotes cardiomyocyte cell cycle re-entry and that this results in functional improvement post myocardial infarction.

```
Sequences
HMGA1
SEQ ID NO: 1
                10 20 30 40
MSESSSKSSQ PLASKQEKDG TEKRGRGRPR KQPPVSPGTA
                50 60 70 80
LVGSQKEPSE VPTPKRPRGR PKGSKNKGAA KTRKTTTTPG
                90 100
RKPRGRPKKL EKEEEEGISQ ESSEEEQ.
HMGA2
SEQ ID NO: 2
                10 20 30 40
MSARGEGAGQ PSTSAQGQPA APAPQKRGRG RPRKQQQEPT
                50 60 70 80
GEPSPKRPRG RPKGSKNKSP SKAAQKKAEA TGEKRPRGRP
                90 100
RKWPQQVVQK KPAQEETEET SSQESAEED.
```

1. A method of promoting cardiomyocyte proliferation in an individual suffering from a structural cardiac muscle defect, comprising
providing cardiomyocytes of at least part of the cardiac muscle of the individual with a high mobility group A (HMGA) protein, to thereby promote proliferation of said cardiomyocytes.

2. The method according to claim 1, wherein said HMGA protein is provided to said cardiomyocytes by systemic or local administration.

3. The method according to claim 1, wherein said HMGA protein is provided to said cardiomyocytes by injection or infusion into the myocardium.

4. The method according to claim 1, wherein said HMGA protein comprises HMGA1, a part of HMGA1 comprising at least amino acid residues 21-89 of SEQ ID NO:1, or a protein that is at least 75% identical to amino acid residues 21-89 of SEQ ID NO:1 over the whole sequence.

5. The method according to claim 1, wherein said HMGA protein is provided to said cardiomyocytes by an expression construct that expresses said HMGA protein in said cardiomyocytes.

6. The method according to claim 5, wherein the expression construct is a viral vector, or a nucleic acid construct.

7. The method according to claim 1, wherein the structural cardiac muscle defect is a congenital heart defect.

8. The method according to claim 1, wherein the structural cardiac muscle defect is hypoplastic left heart syndrome or hypoplastic right heart syndrome.

9. The method according to claim 1, wherein the individual is an adult who suffers from a myocardial infarction or heart failure.

10. An expression construct for functional expression of high mobility group A (HMGA) protein comprising HMGA1, a part of HMGA1 comprising at least amino acid residues 21-89 of SEQ ID NO:1, or a protein that is at least 75% identical to amino acid residues 21-89 of SEQ ID NO:1 over the whole sequence, in cardiomyocytes.

11. A pharmaceutical composition, comprising a high mobility group A (HMGA) protein or the expression construct of claim 10, and
a pharmacologically acceptable excipient.

12. The pharmaceutical composition according to claim 11, wherein the expression construct is a viral vector, or a nucleic acid construct.

13. The pharmaceutical composition according to claim 11, wherein the expression construct is an adenovirus-based vector or an adeno-associated virus (AAV)-based vector.

14. The pharmaceutical composition according to claim 11, wherein the vector is a mRNA-based construct.

15. A method of culturing cardiomyocytes in vitro, comprising
providing cardiomyocytes with a HMGA protein or the expression construct of claim 10, and
culturing said cardiomyocytes.


SEQUENCE LISTING (5 sequences)

SEQ ID NO: 1 - protein, 107 aa, Homo sapiens
MSESSSKSSQPLASKQEKDGTEKRGRGRPRKQPPVSPGTALVGSQKEPSEVPTPKRPRGRPKGSKNKGAAKTRKTTTTPGRKPRGRPKKLEKEEEEGISQESSEEEQ

SEQ ID NO: 2 - protein, 109 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWPQQVVQKKPAQEETEETSSQESAEED

SEQ ID NO: 3 - protein, 10 aa, synthetic construct
RMRRMRRMRR

SEQ ID NO: 4 - DNA, 44 bp, Danio rerio
  source 1..44: mol_type = genomic DNA
taagacacaatgagtgattctggcaaggacacagtggcgactaa

SEQ ID NO: 5 - DNA, 36 bp, synthetic construct
  source 1..36: mol_type = other DNA
taagacacaatgtggcaaggacacagtggcgactaa